(19) United States
(12) Patent Application Publication
(10) Pub. No.: US 2019/0117596 A1
(43) Pub. Date: Apr. 25, 2019

(54) TY-52156 COMPOUNDS FOR THE TREATMENT OF CANCER

(71) Applicant: WAYNE STATE UNIVERSITY, Detroit, MI (US)

(72) Inventors: Meng-Jer Lee, Shelby Township, MI (US); Jiawei Zhao, Detroit, MI (US)

(73) Assignee: WAYNE STATE UNIVERSITY, Detroit, MI (US)

(21) Appl. No.: 16/094,363

(22) PCT Filed: Apr. 28, 2017
(86) PCT No.: PCT/US17/30248

§ 371 Date: Oct. 17, 2018

Related U.S. Application Data
(60) Provisional application No. 62/329,703, filed on Apr. 29, 2016.

(51) Int. Cl. A61K 31/155; C12Q 1/6886; G01N 33/50

(52) U.S. Cl. CPC A61K 31/155; C12Q 1/6886; C12Q 2600/156; C12Q 2600/106; G01N 33/5011

(57) ABSTRACT

TY-52156 compounds for the treatment of lung cancers and cancers mediated by KRAS gene mutations and/or TGF-β/Smad3 signaling are described. TY-52156 compounds antagonize sphingosine-1-phosphate receptor subtype 3 (S1PR3). Methods and kits for selecting subjects for particular treatments and/or clinical trials are also described.

### CROSS-REFERENCE TO RELATED APPLICATION

This application claims priority to U.S. Provisional Patent Application No. 62/329,703 filed on Apr. 29, 2016, which is incorporated by reference in its entirety as if fully set forth herein.

### FIELD OF THE DISCLOSURE

The present disclosure provides TY-52156 compounds for the treatment of lung cancers and cancers mediated by the oncogene KRAS mutation, sphingosine-1-phosphate receptor 3 (S1PR3), and/or TGF-β/Smad3 signaling. TY-52156 compounds antagonize S1PR3. The disclosure also provides selecting an appropriate therapy for a subject diagnosed with cancer and/or selecting subjects for inclusion in clinical trials.

### BACKGROUND OF THE DISCLOSURE

Cancer develops as the result of genetic damage to DNA and epigenetic changes that affect normal functions of the cells such as cell proliferation, apoptosis, and DNA repair. The risk of cancer increases as more damage accumulates.

There are numerous types of cancers. Lung cancer is an example of a malignant tumor that is characterized by uncontrolled cell growth in tissues of the lung. Lung cancer is divided into two main types: small cell lung cancer (SCLC) and non-small cell lung cancer (NSCLC). NSCLCs comprise 85% of all lung cancers.

Lung cancer is the leading cause of cancer death in the United States. Treatments include surgery, chemotherapy, and radiotherapy. NSCLC is generally treated with surgery, while SCLC is generally treated with chemotherapy and radiotherapy. Although advances in the treatment of lung cancer have been made over the last 20 years, the prognosis for patients with advanced lung cancers remains poor.

Oncogenes, when mutated, have the potential to cause normal cells to become cancerous. The Kirsten ras (KRAS) oncogene is an example of an oncogene of the Ras family.

The proteins encoded by the genes of the Ras family are GTPases that play an important role in cell division, cell differentiation, and apoptosis. Oncogenic KRAS mutation represents one of the most prevalent oncogenic drivers of NSCLC and is found in 25% of NSCLC. Unfortunately, lung cancers driven by KRAS mutations are generally refractory to chemotherapy as well as more targeted therapeutic agents.

### SUMMARY OF THE DISCLOSURE

The present disclosure provides use of TY-52156 compounds to treat lung cancer as well as cancers mediated by the oncogene KRAS mutation, sphingosine-1-phosphate receptor 3 (S1PR3), and/or TGF-β/Smad3 signaling. TY-52156 compounds antagonize S1PR3. The disclosure also provides selecting an appropriate therapy for a subject diagnosed with cancer and/or determining whether the subject should be enrolled in a clinical trial.

### BRIEF DESCRIPTION OF THE FIGURES

FIGS. 1A-1H. Up-regulation of S1PR3 in human lung adenocarcinomas. (1A), qPCR quantitation of S1PR3 mRNA in cDNA arrays of human lung adenocarcinoma specimens (OriGene, HLRT101 and HLRT105). **, p<0.01, Student's t test. (1B), qPCR quantitation of S1PR2 mRNA in a cDNA array of human lung cancers (OriGene, HLRT105). **, p<0.01, Student's t test. (1C), HEK293 cells were transfected with S1PR3 or pcDNA vector. Transfected cells were immunostained with anti-S1PR3 (Cayman Chemical) (IMF, left panels). Arrows, nonspecific fluorescent precipitates used for image orientation. Scale bar=33 μm. (1D), anti-S1PR3 staining of human lung adenocarcinoma tumor microarray (Accumax 306). AdC, adenocarcinoma; N, adjacent normal lung tissue. (1E), immunostaining intensity was quantitated with the National Institutes of Health ImageJ software. Data, analyzed with GraphPad Prism 5 software, are shown as mean±S.E. Statistical significance was analyzed by Student's t test. (1F), representative images of anti-S1PR3 staining of human lung adenocarcinoma and the respective adjacent normal lung epithelial tissue. (1G), quantitation of anti-S1PR3 staining of human lung squamous carcinoma microarray (Accumax 306). Data are mean±S.E. Statistical significance was analyzed by Student's t test. (1H), representative images of anti-S1PR3 staining of human lung squamous carcinoma and the respective adjacent normal lung epithelial tissue.

FIGS. 2A-2C. Oncogenic K-Ras mutant stimulates S1PR3 expression. (2A), LSL-K-Ras^{G12D} mice were intratracheally injected with empty adenoviral (Ad-Ctrl) or Ad-Cre particles (1×10⁸ pfu). The development of lung adenocarcinomas (arrows) was analyzed 2 months later. Scale bar=0.5 cm. (2B), K-Ras^{G12D} mice were injected with Ad-Ctrl or Ad-Cre particles. 2 months later, levels of S1PRs in lungs were measured by qPCR analysis. ** and *, p<0.01 and 0.05, respectively. NS, non-statistically significant. n=5, Student's t test. (2C), immunohistochemical staining of S1PR3 in lung specimens from wild-type or K-Ras transgenic mice. Note that levels of S1PR3 are profoundly increased in lung adenocarcinoma of K-Ras transgenic mice (arrows). Scale bar=200 μm.

FIGS. 3A-3F. TGF-β/SMAD3 signaling contributes to oncogenic K-Ras mutant-stimulated S1PR3 up-regulation. (3A), P, potential SMAD3 binding sites in S1PR3 promoter. 0, transcription initiation site. (3B-3D), HEK293 cells were stably transfected with pBabe-K-Ras^{G12V} or pBabe control vector. Levels of total cellular K-Ras (3B), S1PRs (3C), and TGF-β (3D) were measured by qPCR analysis. (3E), HEK293 cells transfected with pBabe-K-Ras^{G12V} or pBabe control vector were incubated with anti-TGF-β (Cell Signaling, antibody number 3711, 10 μg/ml) or irrelevant normal rabbit IgG (10 μg/ml) at 37° C. for 24 h. Levels of S1PR3 were quantitated by qPCR. (3F), HEK293 cells transfected with pBabe-K-Ras^{G12V} or pBabe control vector were treated with or without SB-431542 (SB4) (inhibitor of TGF-β receptor 1, 10 μM) or SIS3 (inhibitor of SMAD3, 2 μM) at 37° C. for 24 h. Levels of S1PR3 were quantitated by qPCR. **, p<0.01, n=3, Student's t test.

FIGS. 4A-4H. TGF-β/SMAD3 signaling axis up-regulates S1PR3. (4A), HBEC2-KT cells were treated with TGF-β (1 ng/ml) for various times. mRNA levels of S1P receptors were measured by qPCR analysis. Data are mean±S.D. of triplicate determinations. *, p<0.05, Student's t test. (4B), protein levels of S1PR3 in TGF-β (1 ng/ml)-treated HBEC2-KT cells. Lower panel, Western blot intensity was quantitated by National Institutes of Health ImageJ. Data (normalized to actin) are mean±S.D. of triplicate determinations. * and **, p<0.05 and 0.01, respectively, Student's t test. (4C), CHO cells were transduced with adenoviral particles (multiplicity of infection of 200) carrying S1PR1, S1PR2, or S1PR3 vector for 20 h as previously described (8). Extracts were blotted with antibody against S1PR3 (Cayman), S1PR2 (Cayman), or S1PR1 (E49) (8). (4D), mRNAs of S1PR3 and TGF-β in minced C57BL/6 mouse lungs (1-2 mm³) infected with adenoviral active TGF-β (Ad-TGF-β, 1×10⁸ pfu/ml) or empty vector (Ad-Ctrl) (37° C., 24 h). **, p<0.01, n=5, Student's t test. (4E), mRNA levels of SphK1 and SphK2 in TGF-β-treated HBEC2-KT cells. (4F) HBEC2-KT cells (2×10⁶ cells in 100-mm dish, 10 ml of cultural medium) were treated with TGF-β (1 ng/ml) for 24 h. Medium was quantitated for S1P, ceramide (Cer), and sphingomyelin (SPM) by LC-MS/MS (29, 46). (4G), HBEC2-KT were pretreated for 30 min with inhibitors. S1PR3 levels were measured by qPCR, following TGF-β treatment (4 h). The following inhibitors were used: SB4, TGF-β receptor I (SB-431542, 10 μM); SIS3, SMAD3 (2 μM); SB2, p38 kinase (SB-203580, 50 nM); BAY, NFκB (BAY11-7085, 10 μM); JII, JNK (JNK inhibitor II, 10 μM). *, p<0.05; dashed line, non-statistical significance; n=3, ANOVA. Each experiment was repeated 2-3 times with similar results. (4H), cells were pretreated for 30 min with inhibitors, followed by stimulation with TGF-β (1 ng/ml). Activation of p38, JNK, and NFκB was measured by Western blotting with phospho-p38 (P-p38), phospho-JNK (P-p54^{JNK} and P-p46^{JNK}), and phospho-IκBα (p-IκBα). Inhibitors used are: SB-203580 (50 nM) for p38 kinase, JNK inhibitor II (10 μM) for JNK, and BAY11-7085 (10 μM) for NFκB.

FIGS. 5A-5D. SMAD3 transactivates S1PR3 promoter. (5A), immunostaining with anti-phospho-SMAD3 in HBEC2-KT treated with or without TGF-β (1 ng/ml, 15 min). Left, fluorescence; right, DAPI nuclear staining. Scale bar=15.2 μm. (5B), ChIP was performed with anti-phopho-SMAD3 or normal IgG in HBEC2-KT treated with or without TGF-β (1 h). *, p<0.05, TGF-β (+)/anti-phospho-SMAD3 versus TGF-β (−)/anti-phospho-SMAD3 (n=3, Student's t test). (5C), HEK293 cells were co-transfected with pGL3 luciferase vector carrying double-stranded P13, P14, or P15 oligonucleotides, pcDNA-SMAD3 or empty pcDNA plasmids, and Renilla luciferase vector (5:5:1). 24 h later, both firefly and Renilla luciferase activities were measured using the Dual-Luciferase Reporter Assay System (Promega). Firefly luciferase activities were normalized to Renilla luciferase activities. (5D), HEK293 cells were co-transfected with pGL3 luciferase vector carrying P14 or scrambled P14 oligonucleotides, pcDNA-SMAD3 or empty pcDNA plasmids, and Renilla luciferase vector (5:5:1). 24 h later, luciferase activities (firefly/Renilla luciferase activity) were measured. **, p<0.01; NS, non-statistical significance; n=3, Student's t test.

FIGS. 6A-6G. S1PR3 regulates growth and lung colonization of lung adenocarcinoma cells. (6A), H1793 cells were stably transfected with sh-S1PR3 or pRS (sh-Ctrl) vector (11, 16). mRNA levels of S1PR3 were quantitated with qPCR analysis. (6B), H1793 cells (1×10⁶ cells), stably transfected with sh-S1PR3 or sh-Ctrl vector, were subcutaneously inoculated in Scid mice. Tumor volume was measured in two dimensions using calipers, and volume was determined using the formula width²×length×0.52 (49). (6C), 4 weeks after inoculation, tumors were removed and weighed. (6D), Scid mice were injected with H1793 cells transfected with sh-S1PR3 or sh-Ctrl vector (1×10⁶ cells) via tail vein route. 28 days later, tumor nodules on lung surface were scored. (6E), representative images of lung injected with H1793-sh-Ctrl and H1793-sh-S1PR3 cells. Arrows, tumor nodules. Scale bar=0.5 cm. (6F), volume of xenograft tumors in athymic nude mice subcutaneously implanted with H1299 cells stably transfected with S1PR3 or control pcDNA vector (1×10⁶ cells) (11, 16). (6G), qPCR quantitation of S1PR3 levels in H1299/pcDNA and H1299/S1PR3 cells. **, p<0.01, n=6, ANOVA.

FIGS. 7A-7E. Inhibition of S1PR3 diminishes lung carcinoma growth. (7A), C57BL/6 mice were subcutaneously inoculated with LLC cells (1×10⁶ cells). 1 week later, mice were intraperitoneally administered with VPC23019 (1.5 mg/kg of body weight) or control vehicle every 3 days. (7B), S1PR1 and S1PR3 levels in Lewis lung carcinoma cells. -ve, PCR reactions were performed without cDNA. **, p<0.01, n=6, ANOVA. (7C), CHO cells were transduced with adenoviral particles carrying S1PR1, S1PR2, S1PR3, or pcDNA control vector. Cells were serum-starved for 24 h. Subsequently, cells were treated with TY-52156 (10 μM) for 10 min, followed by stimulating with S1P (200 nM, 10 min). ERK1/2 activation (p-ERK) was measured by Western blotting analysis. (7D), C57BL/6 mice were subcutaneously inoculated with LLC cells (1×10⁶ cells). 1 week later, mice were intraperitoneally administered with TY-52156 (10 mg/kg of body weight) or control vehicle every 2 days. **, p<0.01, n=6, ANOVA. (7E), tumor weights were measured 24 days after implantation. **, p<0.01, n=6, ANOVA.

FIGS. 8A-AD. TY-52156 treatment significantly inhibits lung adenocarcinoma in LSL-KRas^{G12D} mouse model. Mice were injected with or without Ad-Cre. Lungs were analyzed for adenocarcinoma 2 months later. (8A) LSL-KRas^{G12D} injected with Ad-Cre. (8B) LSL-KRas^{G12D} injected with Ad-Cre. Mice were i.p. injected with TY-52156 (10 mg/kg body weight) every two days. (8C) LSL-KRas^{G12D} mice injected with Ad-Ctrl. (8D) wild-type C57BL/6 mice injected with Ad-Cre. Arrows, lung adenocarcinomas. Scale bar=400 mm.

FIG. 9. Candidate SMAD3 binding elements (SBEs) on the promoter region of S1PR3 gene.

FIGS. 10A-10D. S1PR3 regulates Snai1/E-cadherin signaling pathway. (10A) H1793 cells, stably transfected with sh-S1PR3 or pRS (sh-Ctrl) vector (11, 16). mRNA levels of S1PR3 were quantitated with qPCR analysis. (10B) Cells were treated with TGF-β (1 ng/ml) for indicated times. Protein levels of Snai1 and E-cadherin (CDH1) were measured by Western-blotting analysis. Fold changes (normalized to actin) of Snai1 and CDH1 proteins are shown in lower panels. Data are mean±SD of triplicate determinations. (10C) HBEC2-KT cells were transfected with sh-S1PR3 or pRS (sh-Ctrl) vector, followed by stimulating with or without TGF-β (1 ng/ml, 24 hours). mRNA levels of S1PR3 and Snai1 were quantitated by qPCR analysis. **, p<0.01, n=3, t-test. (10D) Cells were treated with S1P (200 nM) for indicated times. Protein levels of Snai1 and E-cadherin (CDH1) were measured by Western-blotting analysis. Fold changes (normalized to actin) of Snai1 and CDH1 proteins are shown in lower panels. Data are mean±SD of triplicate determinations.

FIG. 11. A549 cells were transfected with sh-S1PR3 or pRS (sh-Ctrl) vector. Cells were treated with or without TGF-β (1 ng/ml, 4 hours). Protein levels of Snai1 and CDH1 were measured by Western-blotting analysis. Fold changes (normalized to actin) of Snai1 and CDH1 proteins are shown in lower panels. ** and *, p<0.01 and 0.05, respectively; n=3, t-test.

FIGS. 12A-12F. S1PR3 activation increases Snai1 mRNA. (12A) H1793 cells stably transfected with sh-Ctrl or sh-S1PR3 vector were stimulated with S1P (200 nM) for indicated times. mRNA levels of Snai1 were quantitated with qPCR analysis. (12B) H1299 cells stably transfected with pCDNA or S1PR3 plasmid were stimulated with S1P (200 nM) for indicated times. mRNA levels of Snai1 were quantitated with qPCR analysis. (12C) H1299 cells were transiently transduced with adenoviral particles carrying S1PR1 or S1PR3 vector. After stimulating with S1P (200 nM, 2 hrs), mRNA levels of Snai1 were quantitated with qPCR analysis. (12D) Levels of S1PR1 and S1PR3 in H1299 cells transiently transduced with adenoviral particles carrying S1PR1 or S1PR3 vector. (12E) H1299 cells stably transfected with S1PR3 were pretreated with or without TY-52156 (10 mM, 30 min), followed by stimulating with S1P (200 nM, 2 hrs). Snai1 levels were quantitated with qPCR analysis. (12F) mRNA levels of S1PR3, Snai1, and E-cadherin (CDH1) in lungs of wild-type or S1PR3 transgenic mice were quantitated with aPCR analysis. Data represent mean±SD of triplicate determinations. * and **, p<0.05 and 0.01, respectively. t-test. Each experiment was repeated at least one time with similar result.

FIGS. 13A-13G. JNK/AP-1 signaling mediates the S1PR3-induced Snai1 up-regulation. (13A) H1793 cells, pre-treated with or without SP600125 (10 μM, 30 min), were stimulated with S1P (200 nM) for 2 hrs. Snai1 mRNA was quantitated by qPCR analysis. (13B) H1793 cells, pre-treated with or without SP600125 (10 μM, 30 min), were stimulated with S1P (200 nM) for indicated times. Levels of Snai1, pp54JNK/pp46JNK, p54JNK, CDH1, and actin were measured by Western-blotting analysis. (13C-13F) Fold changes (normalized to actin) of Snai1, pp54JNK, pp46JNK, and CDH1 proteins. Data are mean±SD of triplicate determinations. (13G) Left panel, H1793 cells stably transfected with sh-Ctrl or sh-S1PR3 were stimulated with S1P (200 nM) for indicated times. ChIP analysis was performed as described in the Materials and Methods section of Example 1. Note that S1P stimulates c-Jun binding to Snai1 promoter in sh-Ctrl, and not in sh-S1PR3 transfected cells. Input, amplification of total input chromatin was used for a loading control. Middle panel, ChIP analysis was performed with normal IgG (nIgG) or c-Jun immunoprecipitates, followed by PCR amplification with AP-1 primer pair. Right panel, qPCR quantitation of ChIP assays with anti-c-Jun. Note that S1P treatment specifically increases the binding of c-Jun to AP-1 site in the promoter region of Snai1. This increased binding was abrogated in S1PR3 knocked-down cells. ** and NS, p<0.01 and non-statistically significant, respectively; S1P, 0.5 hr vs. S1P, 0 hr. (n=3, t-test).

FIGS. 14A-14E. S1P3 regulates the TGF-β-stimulated IL-6 expression. (14A) H1793 lung adenocarcinoma cells were pre-treated for 30 min with inhibitor of TGF-β receptor I (SB4, SB431542, 10 uM) or Smad3 (SIS3, 2 uM), followed by stimulating with TGF-β (1 ng/ml) for 4 hrs. Levels of IL-6 were analyzed by qPCR. TGF-β treatment significantly stimulated IL-6 expression in H1793 cells. The TGF-β-induced IL-6 expression is significantly diminished when S1PR3 were knocked-down (14B) or in the presence of S1PR3 inhibitor CAY10444 (10 μM) (14C). (14D) S1P treatment increased IL-6 expression in H1793 cells. The S1P-induced IL-6 expression is diminished when S1PR3 were knocked-down. (14E) HBEC2-KT normal lung epithelial cells were transduced with adenoviral particles carrying S1PR3 (Ad-S1PR3) or β-galactosidase (Ad-Ctrl, 200 m.o.i.) for overnight, followed by stimulating with or without S1P (200 nM, 4 hrs). mRNA levels of IL-6 were measured by qPCR analysis. Data are mean±SD from a representative experiment (n=3). Each experiment was repeated at least one time with similar results. Data are mean+S.D. (n=3). *, p<0.05; NS, non-statistic significance; t-test.

FIG. 15. Model for TGF-β stimulation of EMT and inflammation via the S1PR3. TGF-β binding to TGF-β receptor results in the up-regulation of S1PR3 via Smad3 activation. Also, TGF-3 stimulation increases SphK1 expression and S1P production. Subsequently, the autocrine S1P/S1PR3 stimulates effectors such as JNK and AP-1. One consequence of the activation of JNK/AP-1 pathway is stimulation of Snai1 expression, leading to E-cadherin suppression. Moreover, activation of S1P/S1PR3 axis stimulates the expression of the pro-inflammatory cytokine IL-6 in lung adenocarcinoma cells.

FIG. 16. An exemplary human K-Ras protein sequence (SEQ ID NO: 1).

### DETAILED DESCRIPTION

Cancer (neoplasia) is characterized by deregulated cell growth and cell division. There are numerous types of cancers. Examples of cancers include acoustic neuroma, adenocarcinoma, astrocytoma, basal cell cancer, bile duct cancer, bladder cancer, brain cancer, breast cancer, bronchogenic cancer, central nervous system cancer, cervical cancer, colon cancer, lung cancer, prostate cancer, ovarian cancer, pancreatic cancer, thyroid cancer, and leukemia.

Lung cancer is a form of cancer in which cells in the lungs become abnormal and multiply uncontrollably to form a tumor. Although most people who develop lung cancer have a history of tobacco smoking, lung cancer can occur in people who have never smoked.

There are two main types of lung cancer: small cell lung cancer (SCLC) and non-small cell lung cancer (NSCLC). This classification is based on the size of the affected cells when viewed under the microscope. NSCLC accounts for 85% of lung cancer, and SCLC accounts for the remaining 15%.

SCLC grows quickly and metastasizes to other tissues, for example the adrenal glands, liver, brain, and bones. In most cases, SCLC has spread beyond the lung at the time of diagnosis. Most people diagnosed with SCLC survive for less than one year. Less than seven percent survive 5 years after diagnosis.

NSCLC are further divided into three main subtypes: adenocarcinoma, squamous cell carcinoma, and large cell lung carcinoma. Adenocarcinoma arises from cells lining the alveoli located throughout the lungs. Squamous cell carcinoma arises from the squamous cells lining the passages leading from the windpipe to the bronchi. Large cell carcinoma are NSCLCs that are neither adenocarcinoma nor squamous cell carcinoma. The 5 year survival rate for people diagnosed with NSCLC is between 11 to 17 percent, and can be higher or lower depending on the subtype and stage of the cancer.

Oncogenes have the potential to transform cells into tumor cells. In tumor cells, oncogenes are mutated and expressed at high level. The KRAS gene is an oncogene that has been associated with lung cancer. The KRAS gene belongs to the Ras family of oncogenes, which also includes the HRAS and NRAS genes. These genes encode GTPases which play an important role in cell division, cell differentiation, and apoptosis. The K-Ras protein, encoded by the KRAS gene is turned on and off by the GTP and GDP molecules. The K-Ras protein is turned on by binding to GTP to transmit signals. The signals instruct the cell to grow and divide or to mature and differentiate. The K-Ras protein is turned off (inactivated) when it converts GTP to GDP. When the K-Ras protein is bound to GDP, it does not relay signals to the cell nucleus.

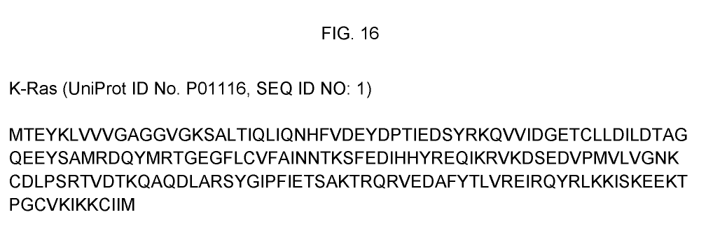

Although genetic, environment, and lifestyle factors contribute to a person's risk for cancer, KRAS gene mutations are more commonly found in nonsmokers with lung cancer than in smokers. At least three KRAS gene mutations are associated with lung cancer. These mutations are somatic, meaning that they are not inherited but are acquired during a person's lifetime, and these mutations are present only in tumor cells. Most of the mutations associated with lung cancer change the amino acid glycine at position 12 or 13 (Gly12 or Gly13) or change the amino acid glutamine at position 61 (Gln61) of the human K-Ras protein sequence (FIG. 16; SEQ ID NO: 1, UniProt ID No. P01116).

These mutations cause the K-Ras protein to be constitutively activated and to direct cells to grow and divide in an uncontrolled manner resulting in tumor formation. Lung cancer develops as a result of these changes occurring in the cells of the lung. Unfortunately, lung cancers with KRAS gene mutations typically indicate poor prognosis and resistance to cancer treatment. Somatic KRAS gene mutations are also found at high rates in leukemia, colon cancer, and pancreatic cancer.

Oncogenic KRAS mutations significantly up-regulate sphingosine-1-phosphate receptor subtype 3 (S1PR3) expression in cultured cells and in animals. S1PR3 is a human gene that encodes a G protein-coupled receptor of the EDG family of receptors. S1PR3 binds sphingosine 1-phosphate (S1P), a lipid signaling molecule, and is therefore a receptor for S1P. S1PR3 is involved in the regulation of angiogenesis and vascular endothelial cell function. Exemplary protein sequences of mammalian S1PR3s can be found at GenBank Accession Numbers: NP_005217.2, AAH68176.1, and JAA32908.1. S1PR3 levels are markedly increased in human lung cancers, and S1PR3 promotes lung cancer progression.

TGF-β/Smad3 signaling pathway also increases the expression of S1PR3 in lung epithelial cells. Activation of S1PR3 in human lung adenocarcinoma cells stimulates c-Jun N-terminal kinase (JNK)/activator protein 1 (AP-1) signaling pathway, which in turn transcriptionally increases Snai1 expression, leading to E-cadherin suppression. S1PR3 knockdown or inhibition diminishes the TGF-β and sphingosine-1-phosphate (S1P) mediated Snai1 up-regulation and E-cadherin suppression. Ectopic expression of S1PR3 results in Snai1 up-regulation and E-cadherin suppression in vitro and in vivo. These results suggest that S1PR3 activity regulates the TGF-β-mediated Snai1 up-regulation and E-cadherin suppression. Since transforming growth factor-β (TGF-β) regulates epithelial-mesenchymal transition which plays a pivotal role in the initiation and progression of cancer, S1PR3 is a target for cancer treatment, particularly those mediated by TGF-β3/Smad3 signaling. Examples of cancers mediated by TGF-β3/Smad3 signaling include prostate cancer, breast cancer, colon cancer, lung cancer, ovarian cancer, and bladder cancer.

The current disclosure describes use of TY-52156 compounds to target S1PR3 as a treatment for lung cancer and cancers mediated by KRAS gene mutations and/or TGF-β/Smad3 signaling. TY-52156 is a highly selective antagonist of S1PR3. It also affects angiogenesis, vascular development, and cardiovascular function such as coronary flow and RhoGTPase activation. Unlike other known antagonists of S1PR3, including antibodies that bind an epitope on the extracellular domain of S1PR3, TY-52156 is a small molecule with the following structural formula:

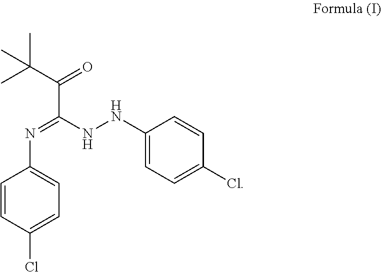 [SMILES: CC(C)(C)C(=O)/C(=N/c1ccc(Cl)cc1)NNc1ccc(Cl)cc1]

The IUPAC name for TY-52156 is 1-(4-chlorophenylhydrazono)-1-(4-chlorophenylamino)-3,3-dimethyl-2-butanone.

“TY-52156 compounds” include TY-52156, derivatives of TY-52156, structurally related compounds disclosed herein, and salts, hydrates, and solvates of TY-52156.

U.S. Pat. No. 8,546,452 describes TY-52156 derivatives and structurally related compounds. These compounds include:
1-(4-chlorophenylhydrazono)-1-(3-fluorophenylamino)-3,3-dimethyl-2-butanone,
1-(4-chlorophenylamino)-1-(4-chlorophenylhydrazono)-3,3-dimethyl-2-butanone,
1-(4-chlorophenylhydrazono)-1-(3,5-difluorophenylamino)-3,3-dimethyl-2-butanone,
1-(4-chlorophenylhydrazono)-1-(3,5-dichlorophenylamino)-3,3-dimethyl-2-butanone,
1-(4-chlorophenylhydrazono)-1-(3,4-dichlorophenylamino)-3,3-dimethyl-2-butanone,
1-(3-chlorophenylhydrazono)-1-(3-fluorophenylamino)-3,3-dimethyl-2-butanone,
1-(4-chlorophenylamino)-1-(3-chlorophenylhydrazono)-3,3-dimethyl-2-butanone,
1-(3-chlorophenylhydrazono)-1-(3,5-dichlorophenylamino)-3,3-dimethyl-2-butanone,
1-(3-chlorophenylhydrazono)-1-(3,4-dichlorophenylamino)-3,3-dimethyl-2-butanone,
1-(3-fluorophenylamino)-1-(phenylhydrazono)-3,3-dimethyl-2-butanone,
1-(4-chlorophenylamino)-1-(phenylhydrazono)-3,3-dimethyl-2-butanone,
1-(3,5-dichlorophenylamino)-1-(phenylhydrazono)-3,3-dimethyl-2-butanone,
1-(3,4-dichlorophenylamino)-1-(phenylhydrazono)-3,3-dimethyl-2-butanone,
1-(3-fluorophenylamino)-1-(4-fluorophenylhydrazono)-3,3-dimethyl-2-butanone,
1-(4-fluorophenylamino)-1-(4-fluorophenylhydrazono)-3,3-dimethyl-2-butanone,
1-(4-chlorophenylamino)-1-(4-fluorophenylhydrazono)-3,3-dimethyl-2-butanone,
1-(4-bromophenylamino)-1-(4-fluorophenylhydrazono)-3,3-dimethyl-2-butanone,
1-(3,5-dichlorophenylamino)-1-(4-fluorophenylhydrazono)-3,3-dimethyl-2-butanone,
1-(3,4-dichlorophenylamino)-1-(4-fluorophenylhydrazono)-3,3-dimethyl-2-butanone,
1-(3-fluorophenylamino)-1-(4-methylphenylhydrazono)-3,3-dimethyl-2-butanone,
1-(4-chlorophenylamino)-1-(4-methylphenylhydrazono)-3,3-dimethyl-2-butanone,
1-(4-methylphenylamino)-1-(4-methylphenylhydrazono)-3,3-dimethyl-2-butanone,
1-(3,5-dichlorophenylamino)-1-(4-methylphenylhydrazono)-3,3-dimethyl-2-butanone,
1-(3,4-dichlorophenylamino)-1-(4-methylphenylhydrazono)-3,3-dimethyl-2-butanone,
1-(4-bromophenylhydrazono)-1-(3-fluorophenylamino)-3,3-dimethyl-2-butanone,
1-(4-bromophenylhydrazono)-1-(4-fluorophenylamino)-3,3-dimethyl-2-butanone,
1-(4-bromophenylhydrazono)-1-(4-chlorophenylamino)-3,3-dimethyl-2-butanone,
1-(4-bromophenylamino)-1-(4-bromophenylhydrazono)-3,3-dimethyl-2-butanone,
1-(4-bromophenylhydrazono)-1-(4-ethynylphenylamino)-3,3-dimethyl-2-butanone,
1-(4-bromophenylhydrazono)-1-(3,5-dichlorophenylamino)-3,3-dimethyl-2-butanone,
1-(4-bromophenylhydrazono)-1-(3,4-dichlorophenylamino)-3,3-dimethyl-2-butanone,
1-(3,4-dichlorophenylhydrazono)-1-(3-fluorophenylamino)-3,3-dimethyl-2-butanone,
1-(4-bromophenylhydrazono)-1-(4-chloro-3-fluorophenylamino)-3,3-dimethyl-2-butanone,
1-(4-chlorophenylamino)-1-(4-ethynylphenylhydrazono)-3,3-dimethyl-2-butanone,
1-(3,4-dichlorophenylhydrazono)-1-(3-ethynylphenylamino)-3,3-dimethyl-2-butanone,
1-(3-chlorophenylamino)-1-(4-ethynylphenylhydrazono)-3,3-dimethyl-2-butanone,
1-(4-chlorophenylhydrazono)-1-(3,4-dichlorophenylamino)-2-hexanone,
1-(4-chlorophenylamino)-1-(4-chlorophenylhydrazono)-3-ethyl-2-pentanone,
1-(4-chlorophenylhydrazono)-1-(4-ethynylphenylamino)-2-hexanone,
1-(4-chlorophenylhydrazono)-1-(4-fluorophenylamino)-3-ethyl-2-pentanone,
1-(4-chlorophenylhydrazono)-1-(3-fluorophenylamino)-4,4-dimethyl-2-pentanone,
1-(4-chlorophenylhydrazono)-1-(4-fluorophenylamino)-4,4-dimethyl-2-pentanone,
1-(4-chlorophenylamino)-1-(4-chlorophenylhydrazono)-2-hexanone,
1-(4-chlorophenylamino)-1-(4-chlorophenylhydrazono)-2-(2-furyl)-2-ethanone,
1-(4-chlorophenylhydrazono)-1-(4-ethynylphenylamino)-2-(2-furyl)-2-ethanone,
1-(4-chlorophenylhydrazono)-1-(4-fluorophenylamino)-2-(2-furyl)-2-ethanone,
1-(4-bromophenylamino)-1-(4-chlorophenylhydrazono)-2-(2-furyl)-2-ethanone,
4-chloro-1-(4-chlorophenylhydrazono)-1-(4-ethynylphenylamino)-3,3-dimethyl-2-butanone,
1-(3-chlorophenylamino)-1-(3,4-dichlorophenylhydrazono)-3,3-dimethyl-2-butanone,
1-(4-chlorophenylamino)-1-(3,4-dichlorophenylhydrazono)-3,3-dimethyl-2-butanone,
1-(3-fluorophenylamino)-1-(4-ethynyl phenyl hydrazono)-3,3-di methyl-2-butanone,
1-(3,4-dichlorophenylhydrazono)-1-(4-ethynylphenylamino)-3,3-dimethyl-2-butanone,
1-(3-chlorophenylamino)-1-(4-chlorophenylhydrazono)-3,3-dimethyl-2-butanone,
1-(4-bromophenylamino)-1-(4-chlorophenylhydrazono)-3,3-dimethyl-2-butanone,
1-(4-chloro-3-fluorophenylamino)-1-(4-chlorophenylhydrazono)-3,3-dimethyl-2-butanone,
1-(4-chlorophenylhydrazono)-1-(4-ethynylphenylamino)-3,3-dimethyl-2-butanone,
1-(3-chlorophenylamino)-1-(4-chlorophenylhydrazono)-3-ethyl-2-pentanone,
1-(4-ethynylphenylamino)-1-(4-ethynylphenylhydrazono)-3,3-dimethyl-2-butanone,
1-(4-chlorophenylamino)-1-(4-chlorophenylhydrazono)-4,4-dimethyl-2-pentanone,
1-(3-chlorophenylamino)-1-(4-chlorophenylhydrazono)-4,4-dimethyl-2-pentanone, and
1-(3-chlorophenylamino)-1-(4-chlorophenylhydrazono)-2-hexanone.

Salts of the TY-52156 compounds disclosed herein include those prepared with inorganic acids such as hydrochloride, hydrobromide, hydroiodide, sulfate, nitrate and phosphate; and those prepared with organic acids such as acetate, trifluoroacetate, oxalate, fumarate, maleate, tartrate, mesylate and tosylate; and the like.

TY-52156 compounds can be formulated into compositions for administration to a subject. For injection, compositions can be formulated as aqueous solutions, such as in buffers including Hanks' solution, Ringer's solution, or physiological saline. The aqueous solutions can contain formulatory agents such as suspending, stabilizing, and/or dispersing agents. Examples of suitable aqueous and non-aqueous carriers, which may be employed in the injectable formulations include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of selected particle size in the case of dispersions, and by the use of surfactants.

Injectable formulations may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like in the compositions.

Alternatively, the composition can be in lyophilized form and/or provided in powder form for constitution with a suitable vehicle, e.g., sterile pyrogen-free water, before use. Lyophilized compositions can include less than 5% water content; less than 4.0% water content; or less than 3.5% water content.

In particular embodiments, the composition can be in a unit dosage form, such as in a suitable diluent in sterile, hermetically sealed ampoules or sterile syringes.

In particular embodiments, in order to prolong the effect of a composition, it is desirable to slow the absorption of the active ingredient(s) following injection. Compositions can be formulated as sustained-release systems utilizing semipermeable matrices of solid polymers containing at least one administration form. Various sustained-release materials have been established and are well known by those of ordinary skill in the art. Sustained-release systems may, depending on their chemical nature, release active ingredients following administration for a few weeks up to over 100 days.

In particular embodiments, delayed absorption can be accomplished by dissolving or suspending the active ingredient(s) in an oil vehicle. In particular embodiments, administration forms can be formulated as depot preparations. Depot preparations can be formulated with suitable polymeric or hydrophobic materials (for example, as an emulsion in an acceptable oil) or ion exchange resins, or as sparingly soluble derivatives, for example, as sparingly soluble salts.

In addition, prolonged absorption of the injectable composition may be brought about by the inclusion of agents which delay absorption such as aluminum monostearate and gelatin.

Injectable depot forms can be made by forming microencapsule matrices of administration forms in biodegradable polymers such as polylactide-polyglycolide. Depending on the ratio of administration form to polymer, and the nature of the particular polymer employed, the rate of administration form release can be controlled. Examples of other biodegradable polymers include poly(orthoesters) and poly(anhydrides). Injectable depot formulations are also prepared by entrapping the active ingredient(s) in liposomes or microemulsions which are compatible with body tissue.

Alternatively, delayed absorption of a composition can be accomplished by the use of a liquid suspension of crystalline or amorphous material having poor water solubility. The rate of absorption of the active ingredient(s) then depends upon its rate of dissolution which, in turn, may depend upon crystal size and crystalline form.

Compositions can also be administered with anesthetics including ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and/or phenazopyridine.

Compositions can also be formulated for oral administration. For ingestion, compositions can take the form of tablets, pills, lozenges, sprays, liquids, and capsules formulated in conventional manners. Ingestible compositions can be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable supplements with improved mouthfeel. U.S. Pat. No. 5,965,162 relates to compositions and methods for preparing comestible units which disintegrate quickly in the mouth.

For administration by inhalation (e.g., nasal or pulmonary), the compositions can be formulated as aerosol sprays for pressurized packs or a nebulizer, with the use of suitable propellants, e.g. dichlorodifluoromethane, trichlorofluoromethane, or dichlorotetra-fluoroethane.

Any composition described herein can advantageously include any other pharmaceutically acceptable carriers which include those that do not produce significantly adverse, allergic, or other untoward reactions that outweigh the benefit of administration, whether for research, prophylactic, and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants. Fillers and excipients are commercially available from companies such as Aldrich Chemical Co., FMC Corp, Bayer, BASF, Alexi Fres, Witco, Mallinckrodt, Rhodia, ISP, and others.

In particular embodiments, the compositions can include, for example, 0.025 μg/mL-5 mg/mL TY-52156 compounds.

The compositions described herein can be used to treat subjects (humans, veterinary animals (dogs, cats, reptiles, birds, etc.) livestock (horses, cattle, goats, pigs, chickens, etc.) and research animals (monkeys, rats, mice, fish, etc.). Subjects in need of a treatment (in need thereof) are subjects diagnosed with lung cancer and/or KRAS gene mutation mediated cancers or TGF-β/Smad3 signaling mediated cancers. Examples of such cancers include lung cancer, colon cancer, pancreatic cancer, bladder cancer, prostate cancer, breast cancer, ovarian cancer, and leukemia. In particular embodiments, the lung cancer is NSCLC.

Treating subjects includes delivering therapeutically effective amounts. Therapeutically effective amounts include those that provide effective amounts, prophylactic treatments and/or therapeutic treatments.

An “effective amount” is the amount of active agent(s) or composition(s) necessary to result in a desired physiological change in vivo or in vitro. Effective amounts are often administered for research purposes. In particular embodiments, effective amounts can be assessed by examining cell growth as determined by MTT and colony formation assays. Cell counting can also be performed to determine cell doubling times and growth rates. Cell viability can be determined by trypan blue exclusion.

A “prophylactic treatment” includes a treatment administered to a subject who does not display signs or symptoms of cancer relapse or metastasis or displays only early signs or symptoms of cancer relapse or metastasis such that treatment is administered for the purpose of diminishing, preventing, or decreasing the risk of developing cancer relapse or metastasis further. Thus, a prophylactic treatment functions as a preventative treatment against cancer relapse or metastasis. In particular embodiments, prophylactic treatments prevent, reduce, or delay cancer relapse or metastasis from a primary tumor site from occurring.

A “therapeutic treatment” includes a treatment administered to a subject who displays symptoms or signs of cancer (initial or relapsed) metastasis and is administered to the subject for the purpose of diminishing or eliminating further signs or symptoms of cancer or metastasis. The therapeutic treatment can reduce, control, or eliminate the presence or activity of cancer or metastasis and/or reduce control or eliminate side effects of cancer or metastasis. In particular embodiments, therapeutic treatments prevent, reduce, or delay further cancer or metastasis from occurring.

In particular embodiments, therapeutically effective amounts provide an anti-cancer effect, through providing an effective amount, a prophylactic treatment and/or a therapeutic treatment. As used herein, an anti-cancer effect refers to a biological effect, which can be manifested by a decrease in tumor volume, a decrease in the number of cancer cells, a decrease in the number of metastases, an increase in life expectancy, or a decrease of various physiological symptoms associated with the cancerous condition. An anti-cancer effect can also be manifested by a decrease in recurrence or an increase in the time before recurrence.

Cancer (medical term: malignant neoplasm) refers to a class of diseases in which a group of cells display uncontrolled growth (division beyond the normal limits), invasion (intrusion on and destruction of adjacent tissues), and sometimes metastasis. “Metastasis” refers to the spread of cancer cells from their original site of proliferation to another part of the body. For solid tumors, the formation of metastasis is a very complex process and depends on detachment of malignant cells from the primary tumor, invasion of the extracellular matrix, penetration of the endothelial basement membranes to enter the body cavity and vessels, and then, after being transported by the blood or lymph, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential.

A “tumor” is a swelling or lesion formed by an abnormal growth of cells (called neoplastic cells or tumor cells). A “tumor cell” is an abnormal cell that divides by a rapid, uncontrolled cellular proliferation and continues to divide after the stimuli that initiated the new division cease. Tumors show partial or complete lack of structural organization and functional coordination with the normal tissue, and usually form a distinct mass of tissue, which may be either benign, pre-malignant or malignant.

For administration, therapeutically effective amounts (also referred to herein as doses) can be initially estimated based on results from in vitro assays and/or animal model studies. Such information can be used to more accurately determine useful doses in subjects of interest. Particularly useful pre-clinical tests include measure of cell growth, cell death, and/or cell viability.

The actual dose amount administered to a particular subject can be determined by a physician, veterinarian, or researcher taking into account parameters such as physical, physiological and psychological factors including target, body weight, stage of cancer, the type of cancer, previous or concurrent therapeutic interventions, idiopathy of the subject, and route of administration.

Exemplary doses can include 0.05 mg/kg to 5.0 mg/kg of the drug disclosed herein. The total daily dose can be 0.05 mg/kg to 30.0 mg/kg of a drug administered to a subject one to three times a day, including administration of total daily doses of 0.05-3.0, 0.1-3.0, 0.5-3.0, 1.0-3.0, 1.5-3.0, 2.0-3.0, 2.5-3.0, and 0.5-3.0 mg/kg/day of administration forms of a drug using 60-minute oral, intravenous or other dosing. In one particular example, doses can be administered QD or BID to a subject with, e.g., total daily doses of 1.5 mg/kg, 3.0 mg/kg, or 4.0 mg/kg of a composition with up to 92-98% wt/v of the compounds disclosed herein.

Additional useful doses can often range from 0.1 to 5 μg/kg or from 0.5 to 1 μg/kg. In other examples, a dose can include 1 μg/kg, 10 μg/kg, 20 μg/kg, 40 μg/kg, 80 μg/kg, 200 μg/kg, 0.1 to 5 mg/kg or from 0.5 to 1 mg/kg. In other examples, a dose can include 1 mg/kg, 10 mg/kg, 20 mg/kg, 40 mg/kg, 80 mg/kg, 200 mg/kg, 400 mg/kg, 450 mg/kg, or more.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

In particular embodiments, the compositions disclosed herein can be used in conjunction with other cancer treatments, such as chemotherapeutic agents, radiation therapy, and/or immunotherapy. The compositions described herein can be administered simultaneously or sequentially with another treatment within a selected time window, such as within 10 minutes, 1 hour, 3 hour, 10 hour, 15 hour, 24 hour, or 48 hour time windows or when the complementary treatment is within a clinically-relevant therapeutic window.

Further, the current disclosure describes a method for determining whether a treatment is appropriate for a subject diagnosed with cancer. As an example, a biological sample from the subject can be screened for the presence of a KRAS gene mutation and/or evidence of TGF-3/Smad3 signaling. In particular embodiments, the presence of the KRAS gene mutation and/or evidence of TGF-β/Smad3 signaling in the sample indicates that treatment with a TY-52156 compound would be appropriate. In particular embodiments, cells from a subject sample can be cultured, and a TY-52156 compound can be administered to the cultured cells to determine if the TY-52156 compound is effective in inhibiting the growth of the cultured cells. As an example, TY-52156 is effective in inhibiting proliferation and colony formation of cultured human lung adenocarcinoma cells exhibiting K-Ras^{G12D} mutation.

Determining whether a treatment is appropriate for a subject includes performing a test to assess whether the subject is more or less likely to respond to a given therapeutic intervention, such as treatment with a TY-52156 compound. Actual response to the therapeutic intervention is not required.

Evidence of TGF-β/Smad3 signaling can be based on increased S1PR3, increased SphK1, increased S1P, increased Snai1, increased interleukin 6, or decreased E-cadherin within cells within the sample. An “increase” or a “decrease” (e.g., up-regulation or down-regulation) can be measured against a relevant control condition as disclosed herein. In particular embodiments, conclusions are drawn based on whether a measure is statistically significantly different or not statistically significantly different from a reference level of a relevant control. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various systems and methods used in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than or equal to 0.05.

Examples of biological samples from a subject include a tissue biopsy sample, a tumor biopsy sample, a blood sample, a serum sample, a saliva sample, a urine sample, or a bronchoalveolar lavage sample.

The current disclosure also includes selecting subjects for enrollment in clinical trials. As an example, a biological sample from the subject can be screened for the presence of a KRAS gene mutation and/or TGF-β/Smad3 signaling. The presence of the KRAS gene mutation and/or TGF-β/Smad3 signaling in the sample could direct the subject for inclusion or exclusion from a clinical trial. A further clinical trial selection step could be based on whether cells from the sample are sensitive to treatment with a TY-52156 compound formation of cultured human lung adenocarcinoma cells exhibiting a K-Ras^{G12D} mutation.

In particular embodiments determining whether a treatment is appropriate or selecting subjects for enrollment in clinical trials can be based on detecting binding between a probe and a relevant target. Probes can include primers, antibodies, binding domains or any other molecule capable of binding a target of interest to indicate a condition.

In particular embodiments, “bind” means that the binding domain associates with the target of interest with a dissociation constant (1(D) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷ M, in particular embodiments of from 10⁻⁸ M to 10⁻¹³ M, or in particular embodiments of from 10⁻⁹ M to 10⁻¹³ M. The term can be further used to indicate that the binding domain does not bind to other biomolecules present, (e.g., it binds to other biomolecules with a dissociation constant (KD) of 10⁻⁴ M or more, in particular embodiments of from 10⁻⁴ M to 1 M).

Antibodies to S1PR3, SphK1, S1P, Snai1, IL-6, and E-cadherin are commercially available from suppliers such as Abcam, R&D Systems, Invitrogen, BioLegend, Santa Cruz Biotechnology, and ThermoFisher. Additional exemplary primers are described and disclosed elsewhere herein.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A method of treating cancer in a subject in need thereof including administering a therapeutically effective amount of a TY-52156 compound to the subject, thereby treating the cancer in the subject.
2. A method of embodiment 1, wherein the TY-52156 compound includes 1-(4-chlorophenylhydrazono)-1-(3-fluorophenylamino)-3,3-dimethyl-2-butanone,
1-(4-chlorophenylamino)-1-(4-chlorophenylhydrazono)-3,3-dimethyl-2-butanone,
1-(4-chlorophenylhydrazono)-1-(3,5-difluorophenylamino)-3,3-dimethyl-2-butanone,
1-(4-chlorophenylhydrazono)-1-(3,5-dichlorophenylamino)-3,3-dimethyl-2-butanone,
1-(4-chlorophenylhydrazono)-1-(3,4-dichlorophenylamino)-3,3-dimethyl-2-butanone,
1-(3-chlorophenylhydrazono)-1-(3-fluorophenylamino)-3,3-dimethyl-2-butanone,
1-(4-chlorophenylamino)-1-(3-chlorophenylhydrazono)-3,3-dimethyl-2-butanone,
1-(3-chlorophenylhydrazono)-1-(3,5-dichlorophenylamino)-3,3-dimethyl-2-butanone,
1-(3-chlorophenylhydrazono)-1-(3,4-dichlorophenylamino)-3,3-dimethyl-2-butanone,
1-(3-fluorophenylamino)-1-(phenylhydrazono)-3,3-dimethyl-2-butanone,
1-(4-chlorophenylamino)-1-(phenylhydrazono)-3,3-dimethyl-2-butanone,
1-(3,5-dichlorophenylamino)-1-(phenylhydrazono)-3,3-dimethyl-2-butanone,
1-(3,4-dichlorophenylamino)-1-(phenylhydrazono)-3,3-dimethyl-2-butanone,
1-(3-fluorophenylamino)-1-(4-fluorophenylhydrazono)-3,3-dimethyl-2-butanone,
1-(4-fluorophenylamino)-1-(4-fluorophenylhydrazono)-3,3-dimethyl-2-butanone,
1-(4-chlorophenylamino)-1-(4-fluorophenylhydrazono)-3,3-dimethyl-2-butanone,
1-(4-bromophenylamino)-1-(4-fluorophenylhydrazono)-3,3-dimethyl-2-butanone,
1-(3,5-dichlorophenylamino)-1-(4-fluorophenylhydrazono)-3,3-dimethyl-2-butanone,
1-(3,4-dichlorophenylamino)-1-(4-fluorophenylhydrazono)-3,3-dimethyl-2-butanone,
1-(3-fluorophenylamino)-1-(4-methylphenylhydrazono)-3,3-dimethyl-2-butanone,
1-(4-chlorophenylamino)-1-(4-methylphenylhydrazono)-3,3-dimethyl-2-butanone,
1-(4-methylphenylamino)-1-(4-methylphenylhydrazono)-3,3-dimethyl-2-butanone,
1-(3,5-dichlorophenylamino)-1-(4-methylphenylhydrazono)-3,3-dimethyl-2-butanone,
1-(3,4-dichlorophenylamino)-1-(4-methylphenylhydrazono)-3,3-dimethyl-2-butanone,
1-(4-bromophenylhydrazono)-1-(3-fluorophenylamino)-3,3-dimethyl-2-butanone,
1-(4-bromophenylhydrazono)-1-(4-fluorophenylamino)-3,3-dimethyl-2-butanone,
1-(4-bromophenylhydrazono)-1-(4-chlorophenylamino)-3,3-dimethyl-2-butanone,
1-(4-bromophenylamino)-1-(4-bromophenylhydrazono)-3,3-dimethyl-2-butanone,
1-(4-bromophenylhydrazono)-1-(4-ethynylphenylamino)-3,3-dimethyl-2-butanone,
1-(4-bromophenylhydrazono)-1-(3,5-dichlorophenylamino)-3,3-dimethyl-2-butanone,
1-(4-bromophenylhydrazono)-1-(3,4-dichlorophenylamino)-3,3-dimethyl-2-butanone,
1-(3,4-dichlorophenylhydrazono)-1-(3-fluorophenylamino)-3,3-dimethyl-2-butanone,
1-(4-bromophenylhydrazono)-1-(4-chloro-3-fluorophenylamino)-3,3-dimethyl-2-butanone,
1-(4-chlorophenylamino)-1-(4-ethynylphenylhydrazono)-3,3-dimethyl-2-butanone,
1-(3,4-dichlorophenylhydrazono)-1-(3-ethynylphenylamino)-3,3-dimethyl-2-butanone,
1-(3-chlorophenylamino)-1-(4-ethynylphenylhydrazono)-3,3-dimethyl-2-butanone,
1-(4-chlorophenylhydrazono)-1-(3,4-dichlorophenylamino)-2-hexanone,
1-(4-chlorophenylamino)-1-(4-chlorophenylhydrazono)-3-ethyl-2-pentanone,
1-(4-chlorophenylhydrazono)-1-(4-ethynylphenylamino)-2-hexanone,
1-(4-chlorophenylhydrazono)-1-(4-fluorophenylamino)-3-ethyl-2-pentanone,
1-(4-chlorophenylhydrazono)-1-(3-fluorophenylamino)-4,4-dimethyl-2-pentanone,
1-(4-chlorophenylhydrazono)-1-(4-fluorophenylamino)-4,4-dimethyl-2-pentanone,
1-(4-chlorophenylamino)-1-(4-chlorophenylhydrazono)-2-hexanone,
1-(4-chlorophenylamino)-1-(4-chlorophenylhydrazono)-2-(2-furyl)-2-ethanone,
1-(4-chlorophenylhydrazono)-1-(4-ethynylphenylamino)-2-(2-furyl)-2-ethanone,
1-(4-chlorophenylhydrazono)-1-(4-fluorophenylamino)-2-(2-furyl)-2-ethanone,
1-(4-bromophenylamino)-1-(4-chlorophenylhydrazono)-2-(2-furyl)-2-ethanone,
4-chloro-1-(4-chlorophenylhydrazono)-1-(4-ethynylphenylamino)-3,3-dimethyl-2-butanone,
1-(3-chlorophenylamino)-1-(3,4-dichlorophenylhydrazono)-3,3-dimethyl-2-butanone,
1-(4-chlorophenylamino)-1-(3,4-dichlorophenylhydrazono)-3,3-dimethyl-2-butanone,
1-(3-fluorophenylamino)-1-(4-ethynylphenylhydrazono)-3,3-dimethyl-2-butanone,
1-(3,4-dichlorophenylhydrazono)-1-(4-ethynylphenylamino)-3,3-dimethyl-2-butanone,
1-(3-chlorophenylamino)-1-(4-chlorophenylhydrazono)-3,3-dimethyl-2-butanone,
1-(4-bromophenylamino)-1-(4-chlorophenylhydrazono)-3,3-dimethyl-2-butanone,
1-(4-chloro-3-fluorophenylamino)-1-(4-chlorophenylhydrazono)-3,3-dimethyl-2-butanone,
1-(4-chlorophenylhydrazono)-1-(4-ethynylphenylamino)-3,3-dimethyl-2-butanone,
1-(3-chlorophenylamino)-1-(4-chlorophenylhydrazono)-3-ethyl-2-pentanone,
1-(4-ethynylphenylamino)-1-(4-ethynylphenylhydrazono)-3,3-dimethyl-2-butanone,
1-(4-chlorophenylamino)-1-(4-chlorophenylhydrazono)-4,4-dimethyl-2-pentanone,
1-(3-chlorophenylamino)-1-(4-chlorophenylhydrazono)-4,4-dimethyl-2-pentanone, and
1-(3-chlorophenylamino)-1-(4-chlorophenylhydrazono)-2-hexanone.
   3. The method of embodiment 1 or 2, wherein the TY-52156 compound includes a compound of structural formula:

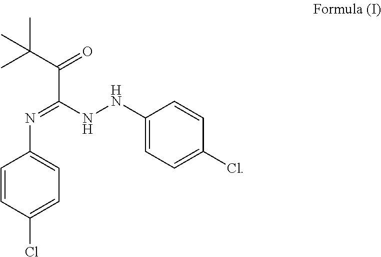 [SMILES: CC(C)(C)C(=O)/C(=N/c1ccc(Cl)cc1)NNc1ccc(Cl)cc1]

4. The method of any of embodiments 1-3, wherein the cancer is mediated by a KRAS mutation and/or TGF-β/Smad3 signaling.
5. The method of any of embodiments 1-4, wherein the cancer is lung cancer, breast cancer, colon cancer, pancreatic cancer, bladder cancer, ovarian cancer, prostate cancer, or leukemia.
6. The method of embodiment 4, wherein the cancer mediated by a KRAS mutation is lung cancer, colon cancer, pancreatic cancer, or leukemia.
7. The method of embodiment 5 or 6, wherein the lung cancer is non-small cell lung cancer (NSCLC).
8. The method of embodiment 5 or 6, wherein the lung cancer is adenocarcinoma, squamous cell carcinoma, or large cell lung carcinoma.
9. The method of embodiment 5 or 6, wherein the lung cancer is small cell lung cancer.
10. A method for determining whether therapy including a TY-52156 compound is appropriate for a subject diagnosed with cancer, wherein the method includes obtaining a biological sample from the subject; testing the biological sample for the presence of a KRAS gene mutation and/or evidence of TGF-β/Smad3 signaling; and determining that therapy with a TY-52156 compound is appropriate for the subject if the KRAS gene mutation and/or evidence of TGF-β/Smad3 signaling is present.
11. The method of embodiment 10, wherein the evidence of TGF-β/Smad3 signaling includes increased S1PR3, increased SphK1, increased S1P, increased Snai1, increased interleukin 6, or decreased E-cadherin.
12. The method of embodiment 10 or 11, wherein the method further includes confirming therapy with a TY-52156 compound is appropriate by culturing cells of the biological sample and administering a TY-52156 compound to the cultured cells wherein the TY-52156 compound inhibits cell growth in the culture.
13. The method of any of embodiments 10-12 wherein the TY-52156 compound includes a compound of embodiment 2 or 3.
14. The method of any of embodiments 10-13, wherein the biological sample is a tissue biopsy sample, a tumor biopsy sample, a blood sample, a serum sample, a saliva sample, a urine sample, or a bronchoalveolar larvage sample.
15. The method of any of embodiments 10-14, wherein the cancer is lung cancer, colon cancer, pancreatic cancer, or leukemia.
16. The method of any of embodiments 10-14, wherein the cancer is lung cancer.
17. The method of any of embodiments 10-14, wherein the lung cancer is NSCLC.
18. The method of any of embodiments 10-14, wherein the lung cancer is adenocarcinoma, squamous cell carcinoma, or large cell lung carcinoma.
19. The method of any of embodiments 10-14, wherein the lung cancer is small cell lung cancer.
20. A method for determining whether a subject should be enrolled in a clinical trial aimed at examining the efficacy of a therapeutic treatment against a cancer including obtaining a biological sample from the subject; testing the biological sample for the presence of a KRAS gene mutation and/or evidence of TGF-β/Smad3 signaling; and determining that the subject should be enrolled in the clinical trial if the KRAS gene mutation and/or evidence of TGF-β/Smad3 signaling is present.
21. A method for determining whether a subject should be enrolled in a clinical trial aimed at examining the efficacy of a therapeutic treatment including obtaining a biological sample from the subject; testing the biological sample for the presence of a KRAS gene mutation and/or evidence of TGF-β/Smad3 signaling; and determining that the subject should not be enrolled in the clinical trial if the KRAS gene mutation and/or evidence of TGF-β/Smad3 signaling is present.
22. The method of embodiment 20 or 21, wherein the evidence of TGF-β/Smad3 signaling includes increased S1PR3, increased SphK1, increased S1P, increased Snai1, increased interleukin 6, or decreased E-cadherin.
23. The method of any of embodiments 20-22 wherein the clinical trial examines the efficacy of a therapeutic cancer treatment utilizing a TY-52156 compound.
24. The method of embodiment 23 wherein the TY-52156 compound includes a compound of embodiment 2 or 3.
25. The method of any of embodiments 20-24, wherein the biological sample is a tissue biopsy sample, a tumor biopsy sample, a blood sample, a serum sample, a saliva sample, a urine sample, or a bronchoalveolar larvage sample.
26. The method of any of embodiments 20-24, wherein the cancer is lung cancer, colon cancer, pancreatic cancer, or leukemia.
27. The method of any of embodiments 20-24, wherein the cancer is lung cancer.
28. The method of any of embodiments 20-24, wherein the lung cancer is NSCLC.
29. The method of any of embodiments 20-24, wherein the lung cancer is adenocarcinoma, squamous cell carcinoma, or large cell lung carcinoma.
30. The method of any of embodiments 20-24, wherein the lung cancer is small cell lung cancer.
31. A method of determining whether a cancer treatment is appropriate and treating cancer in a subject in need thereof, said method including:

obtaining a sample from the subject;

detecting and measuring one or more of S1PR3, SphK1, S1P, Snai1, IL-6, and E-cadherin in the sample;

determining that the cancer treatment is appropriate when S1PR3 is increased, SphK1 is increased, S1P is increased, Snai1 is increased, IL-6 is increased, and/or E-cadherin is decreased, relative to a control sample; and

administering the cancer treatment comprising an effective amount of a TY-52156 compound to the subject;

thereby determining whether a cancer treatment is appropriate and treating cancer in the subject in need thereof.

32. The method of embodiment 31 wherein the detecting and measuring includes contacting the sample with a probe for S1PR3, a probe for SphK1, a probe for S1P, a probe for Snai1, a probe for IL-6, and/or a probe for E-cadherin; and measuring binding between the probe for S1PR3 and S1PR3, the probe for SphK1 and SphK1, the probe for S1P and S1P, the probe for Snai1 and Snai1, the probe for IL-6 and IL-6, and/or the probe for E-cadherin and E-cadherin.
33. The method of embodiment 32 wherein the probe for S1PR3, the probe for SphK1, the probe for S1P, the probe for Snai1, the probe for IL-6, and/or the probe for E-cadherin are antibodies.
34. The method of any of embodiments 31-33 wherein the cancer includes lung cancer, colon cancer, pancreatic cancer or leukemia.
35. A method of embodiment 34 wherein the cancer includes lung cancer.
36. A method of embodiment 35 wherein the lung cancer includes NSCLC.
37. A method of embodiment 35 wherein the lung cancer includes adenocarcinoma, squamous cell carcinoma, or large cell lung carcinoma.
38. A method of determining whether a cancer treatment is appropriate and treating cancer in a subject in need thereof, said method including:

obtaining a sample from the subject;

detecting whether the KRAS gene is mutated in the sample;

determining that the cancer treatment is appropriate when a mutation in the KRAS gene is detected; and

administering the cancer treatment comprising an effective amount of a TY-52156 compound to the subject;

thereby determining whether the cancer treatment is appropriate and treating cancer in the subject in need thereof.

39. A method of embodiment 38 wherein the KRAS gene mutation changes the amino acid at position 12, 13, or 61 of the human K-Ras protein sequence.
40. A method of embodiment 38 or 39 wherein the cancer includes lung cancer, colon cancer, pancreatic cancer or leukemia.
41. A method of embodiment 40 wherein the cancer includes lung cancer.
42. A method of embodiment 41 wherein the lung cancer includes NSCLC.
43. A method of embodiment 41 wherein the lung cancer includes adenocarcinoma, squamous cell carcinoma, or large cell lung carcinoma.
44. A method of detecting TGF-β/Smad3 signalling in a subject, said method including:

obtaining a sample from the subject;

contacting the sample with a probe for S1PR3, a probe for SphK1, a probe for S1P, a probe for Snai1, a probe for IL-6, and/or a probe for E-cadherin; and

measuring binding between the probe for S1PR3 and S1PR3, the probe for SphK1 and SphK1, the probe for S1P and S1P, the probe for Snai1 and Snai1, the probe for IL-6 and IL-6, and/or the probe for E-cadherin and E-cadherin;

wherein TGF-β/Smad3 signalling is detected when S1PR3 is increased, SphK1 is increased, S1P is increased, Snai1 is increased, IL-6 is increased, and/or E-cadherin is decreased, relative to a control sample.

45. The method of embodiment 44 wherein the probe for S1PR3, the probe for SphK1, the probe for S1P, the probe for Snai1, the probe for IL-6, and/or the probe for E-cadherin are antibodies.
46. A method of detecting S1PR3, SphK1, S1P, Snai1, IL-6, and/or E-cadherin in a subject, said method including:

obtaining a sample from the subject;

contacting the sample with a probe for S1PR3, a probe for SphK1, a probe for S1P, a probe for Snai1, a probe for IL-6, and/or a probe for E-cadherin; and

measuring binding between the probe for S1PR3 and S1PR3, the probe for SphK1 and SphK1, the probe for S1P and S1P, the probe for Snai1 and Snai1, the probe for IL-6 and IL-6, and/or the probe for E-cadherin and E-cadherin.

47. The method of embodiment 46 wherein the probe for S1PR3, the probe for SphK1, the probe for S1P, the probe for Snai1, the probe for IL-6, and/or the probe for E-cadherin are antibodies.

### Example 1

The TGF-β/SMAD3 Pathway Stimulates Sphingosine-1 Phosphate Receptor 3 Expression: Implication of Sphingosine-1 Phosphage Receptor 3 in Lung Adenocarcinoma Progression.

Introduction. Sphingosine-1-phospahte (S1P) is a serum-borne bioactive lipid mediator, which is generated by two sphingosine kinase isozymes, SphK1 and SphK2, using sphingosine as the substrate (1). S1P functions as an extracellular ligand or intracellular lipid mediator (2-5), and regulates various physiological and pathophysiological functions (5-8). When S1P is functioning as an extracellular ligand, its activities are mediated by the S1P family of G protein-coupled receptors (S1PR1-S1PR5) (2, 9-11). Several lines of evidence suggest that S1P-mediated signaling pathways are closely linked to the tumorigenesis of various human cancers (12-16). However, the pathological link between the SiP-mediated signaling pathways and human lung adenocarcinoma is poorly understood. Levels of sphingosine-1 phosphate receptor 3 (S1PR3) are significantly increased in cultured human lung adenocarcinoma cell lines (16). Moreover, the S1PR3-activated signaling pathways play an important role in promoting the progression and invasiveness of human lung adenocarcinoma cells (11, 16).

TGF-β activates multiple signaling pathways to regulate various tumorigenic processes. For example, TGF-β regulates epithelial-mesenchymal transition, which is a critical process in cancer initiation and progression (17-20). Also, TGF-β stimulates the production of inflammatory cytokines in tumor microenvironments (21), and promotes tumor progression through extracellular matrix remodeling, cell adhesion, migration, and immune tolerance (17, 22, 23). Upon TGF-β ligation, TGF-β receptors phosphorylate SMAD (homolog of mothers against decapentaplegic) signaling molecules, leading to the nuclear translocation of SMADs. The nucleus-localized SMADs interact with specific transcriptional activators and repressors and regulate the expression of tumorigenic genes (24). In addition, TGF-β activates SMAD-independent pathways such as MAPK, JNK, NFKB, Ras/Raf/ERK, and Rho kinase pathways in a cell type-dependent manner (24, 25). Although both SMAD and non-SMAD pathways were reported to be involved in tumorigenic process, the mechanistic details remain to be elucidated.

Previous studies have suggested the cross-talk between TGF-β and S1P signaling pathways. TGF-β was shown to activate SphK1 and stimulate the production of S1P (26), which may be involved in extracellular matrix deposition and fibrosis. On the other hand, S1P transactivates the TGF-β pathway and regulates several TGF-β-mediated physiological and pathological functions (27, 28). Thus, a better understanding of the cross-talk between the S1P- and TGF-β mediated signaling pathways is expected to open new perspectives for the treatment of TGF-β-triggered pathologies such as inflammation, fibrosis, and cancer.

In the present Example, it is demonstrated that levels of S1PR3 are significantly increased in human lung adenocarcinoma specimens. Mechanistically, the data show that the TGF-β/SMAD 3 signaling pathway contributes to S1PR3 up-regulation in lung adenocarcinomas. Moreover, the Example shows that S1PR3 represents a novel therapeutic target for the treatment of human lung cancers.

Methods. Reagents. Sphingosine-1 phosphate (Biomol) and VPC23019 (Cayman Chemical) were prepared as micelles by sonicating in aqueous solution of fatty acid-free bovine serum albumin (0.4 mg/ml, Sigma). TY-52156 was chemically synthesized as described (39). TGF-β was from R&D Systems. Anti-S1PR3 and anti-phospho-SMAD3 were from Cayman and Abcam, respectively. Snai1, E-cadherin, phospho-JNK, JNK, and actin antibodies were from Cell Signaling. SB-431542 and SIS3 were purchased from Sigma. Unless specified, other reagents are from Sigma.

Cell Cultures. Immortalized normal human lung epithelial cells (HBEC2-KT and HBEC3-KT) were cultured using keratinocyte-serum free medium (Invitrogen) (75). H1793 human lung adenocarcinoma cells were cultured using HITES medium (RPMI 1640 medium supplemented with hydrocortisone (10 nM), insulin (5 μg/ml), transferrin (100 μg/ml), 17 [3-estradiol (10 nM), sodium selenite (30 nM), and 5% fetal bovine serum) (75). H1299 and mouse Lewis lung carcinoma cells were cultured essentially as previously described (16). Cells were cultured in a humidified atmosphere of 5% CO₂ at 37° C.

Real-time PCR Analysis. Total RNA was isolated using TRIzol reagent (Invitrogen) and was reverse-transcribed with an oligo(dT) primer (Promega) by Moloney Murine Leukemia Virus (M-MLV) Reverse Transcriptase (Promega) for first-strand cDNA synthesis. For real-time PCR quantitation, 50 ng of reversely transcribed cDNAs were amplified with the ABI 7500 system (Applied Biosystems) in the presence of TaqMan DNA polymerase. The qPCR reaction was performed by using a universal PCR Master Mix (Applied Biosystems) according to the manufacturer's instructions. The sense and antisense primers used for qPCR analysis are: human and mouse S1PR1,

sense, 5′-ATCATGGCTGGAACTGCATCA-3′ (SEQ ID NO: 2),
antisense, 5′-CGAGTCCTGACCAAGGAGTAGAT-3 (SEQ ID NO: 3);
human and mouse S1PR2,
sense, 5′-CAGACGCTAGCCCTGCTCAAGA-3′ (SEQ ID NO: 4),
antisense, 5′-TAGTGGGCTTTGTAGAGGA-3′ (SEQ ID NO: 5);
human and mouse S1PR3,
sense, 5′-ACAACCGCATGTACTTTTTCAT-3′ (SEQ ID NO: 6),
antisense, 5′-TACTGCCCTCCCTGAGGAACCA-3′ (SEQ ID NO: 7);
human S1PR4,
sense, 5′-CGGCCATCTTCCGCCTGGTG-3′ (SEQ ID NO: 8),
antisense, 5′-TGCCCCGCAGGTACTCCTGG-3′ (SEQ ID NO: 9);
human S1PR5,
sense, 5′-GGCGCGCACCTGTCCTGTAC-3′ (SEQ ID NO: 10),
antisense, 5′-TCGGGTCTCTGCCGCAGGAG-3′ (SEQ ID NO: 11);
human and mouse SphK1,
sense, 5′-AAACCCCTGTGTAGCCTCCC-3′ (SEQ ID NO: 12),
antisense, 5′-AGCAGGTTCATGGGTGACAG-3′ (SEQ ID NO: 13);
human SphK2,
sense, 5′-GCACAGCAACAGTGAGCA-3′ (SEQ ID NO: 14),
antisense, 5′-GAGCCTGAGTGAGTGGGA-3′ (SEQ ID NO: 15);
porcine TGF-β,
sense, 5′-GCACGTGGAGCTATACCAGAA-3′ (SEQ ID NO: 16),
antisense, 5′-CATCAAAGGACAGCCACTCC-3′ (SEQ ID NO: 17);
human GAPDH,
sense, 5′-GAAGGTGAAGGTCGGAGT-3′ (SEQ ID NO: 18),
antisense, 5′-GAAGATGGTGATGGGTTTC-3′ (SEQ ID NO: 19);
mouse GAPDH,
sense, 5′-CACCTTCGATGCCGGGGCTG-3′ (SEQ ID NO: 20),
antisense, 5′-GGCCATGAGGTCCACCACCC-3′ (SEQ ID NO: 21);
human Snai1,
sense, 5′-GAGGCGGTGGCAGACTAG-3′ (SEQ ID NO: 22);
antisense, 5′-GACACATCGGTCAGACCAG-3′ (SEQ ID NO: 23);
mouse Snai1,
sense, 5′-CCACTGCAACCGTGCTTTT-3′ (SEQ ID NO: 24); and
antisense, 5′-TCTTCACATCCGAGTGGG-3′ (SEQ ID NO: 25)

cDNA array analysis of mRNA levels of S1PR3 was performed using TissueScan qPCR arrays (HLRT101 and HLRT105, OriGene) following the manufacturer's instructions. The results of adenocarcinomas were extracted, and then analyzed by Student's t test.

Immunofluorescence Microscopy. Cells were fixed with 4% paraformaldehyde for 30 min, followed by permeabilization with PBS containing 0.05% Triton X-100. After washing three times with PBS, cells were incubated with primary antibody at room temperature overnight. Cells were then washed three times with PBS, and incubated with FITC-conjugated secondary antibody for 1 h. Fluorescence images were captured by the Leica TCS SP5 confocal system (Leica, Wetzlar, Germany).

Immunohistochemical Staining. Human lung carcinoma tumor microarray (TMA) was purchased from Accumax (Accumax 306). Immunohistochemical staining was performed using VECTASTAIN ABC kit (Vector Laboratories, catalog number PK-6200) following the manufacturer's instructions. Briefly, TMA sections were deparaffinized and dehydrated. Antigen retrieval was performed by microwave irradiation (two cycles of 5 min each) in 10 mM citrate buffer (pH 6.0). TMA was incubated with rabbit polyclonal S1P3 antibody (1:200, Cayman) for 60 min, and then with biotinylated secondary antibody solution for 30 min and VECTASTAIN ABC Reagent for 30 min at room temperature. Subsequently, sections were incubated with peroxidase substrate (ImmPACT DAB (3,3′-diaminobenzidine), Vector Laboratories, catalog number SK-4105) until the desired stain intensity develops. Levels of S1PR3 were visualized by light microscopy (Leica DM13000B).

Western Blotting Analysis. Protein extraction and Western blotting were performed as described (11). Briefly, cells were collected in ice-cold PBS using cell scrapers followed by centrifugation (500×g, 5 min). Cell extracts were prepared with radioimmunoprecipitation assay buffer (10 mM Tris-HCl, pH 7.4, 150 mM NaCl, 5 mM EDTA, 1% Triton X-100, 1% sodium deoxycholate, 0.1% SDS) containing protease inhibitors (Calbiochem) with constant agitation at 4° C. for 30 min. After centrifugation at 15,000×g for 20 min, supernatant was collected and protein concentration was measured using a bicinchoninic acid protein assay kit with BSA as standard. 50 μg of protein extracts were dissolved in 2× Laemmli sample buffer, heated at 95° C. for 5 min, and resolved on a 10% SDS-PAGE gel. After electrophoresis, gels were transferred to nitrocellulose membranes. Subsequently, membranes were blocked in 5% nonfat dry milk (Lab Scientific) in TBST buffer (20 mM Tris-HCl, pH 7.4, 500 mM NaCl, and 0.05% Tween 20). Membranes were washed and incubated with indicated primary antibodies (1:1000 dilution) on a rotary shaker at 4° C. overnight. The blots were then incubated with peroxidase-conjugated secondary antibody for 1 h at room temperature and developed with enhanced chemiluminescent reagent (Thermo Scientific).

ChIP Analysis. The ChIP assay was performed using Pierce Agarose ChIP Kit, following the manufacturer's instructions. Briefly, 1×10⁷ cells were cross-linked with 1% formaldehyde for 10 min. Following the addition of glycine quenching solution, cells were scraped and resuspended in 1×PBS with protease inhibitor cocktails (Calbiochem). Cells were then lysed in lysis buffer, and nuclear lysates were treated with micrococcal nuclease. Lysates were immunoprecipitated with anti-phospho-SMAD3 (Thermo Scientific) at 4° C. overnight. Immunoprecipitation with irrelevant normal IgG was used as a control.

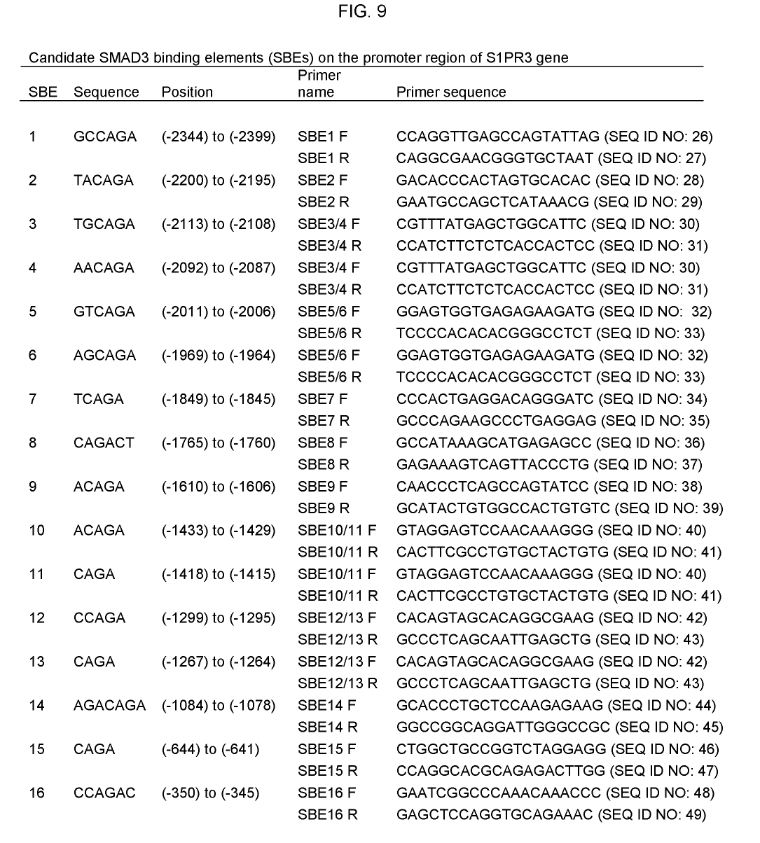

Immune complexes were isolated with protein A/G-Sepharose beads at 4° C. for 1 h. After washings, DNA fragments contained in immune complexes were purified, and then amplified by qPCR reactions. Sequences of primer pairs used for ChIP assay of SMAD3 binding to S1PR3 promoter are shown in FIG. 9 (SEQ ID NOs: 26-49). Pre-designed primer pairs used for ChIP assay of AP-1 binding to Snai1 promoter was purchased from Qiagen (GPH1008503(+)01A).

Luciferase Reporter Assay. Oligonucleotides of candidate. SMAD3 binding sites in the S1PR3 promoter region were synthesized, with an overhanging NheI and SacI restriction site sequence at the 5′-end and 3′-end, respectively, of the antisense strand. Synthesized oligonucleotides are:

P13 sense,
(SEQ ID NO: 50)
5′-GTCAGCAGGCAGAGTCACTTGC-3′;
P13 antisense,
(SEQ ID NO: 51)
5′-CTAGGCAAGTGACTCTGCCTGCTGACAGCT-3′;
P14 sense,
(SEQ ID NO: 52)
5′-GGGCAAAAGACAGAAAGTAACC-3′;
P14 antisense,
(SEQ ID NO: 53)
5′-CTAGGGTTACTTTCTGTCTTTTGCCCAGCT-3′;
P15 sense,
(SEQ ID NO: 54)
5′-GTGCACCAG CAGAGGCTGGGGC-3′;
P15 antisense,
(SEQ ID NO: 55)
5′-CTAGGCCCCAGCCTCTGCTGGTGCACAGCT-3′;
Scramble P14 sense,
(SEQ ID NO: 56)
5′-GGGCAAATGGCGAAAAGTAACC-3′;
Scramble P14 antisense,
(SEQ ID NO: 57)
5′-CTAGGGTTACTTTTCGCCATTTGCCCAGCT-3′.

Equimolar amounts of sense and antisense oligonucleotides were mixed at 95° C. for 5 min, followed by cooling to room temperature. Annealed double-strand oligonucleotides were ligated with NheI- and SacI-digested pGL3-promoter luciferase reporter vector (Promega). Recombinant luciferase vectors were verified by DNA sequencing.

HEK293 cells were co-transfected with recombinant pGL3 luciferase vector, pcDNA-SMAD3 (47) or empty pcDNA plasmids, and pRL-null vector (Promega) carrying the Renilla luciferase gene (5:5:1) by using Lipofectamine 2000 reagent (Life Technologies). 24 h after transfection, both firefly and Renilla luciferase activities were measured with the Dual-Luciferase Reporter Assay System (Promega) using a SpectraMax M3 Multi-mode Microplate Reader (Molecular Devices). Firefly luciferase activities (M1) were normalized to Renilla luciferase activities (M2).

Sphingolipid Measurement by LC-MS/MS. Sphingolipids were extracted from culture medium as previously described (29, 46). Samples were filtered through 0.45-μm nylon filters directly into auto sampler vials for LC-MS/MS analysis. Reverse phase HPLC was performed using BDS HYPERSIL C8 columns (100×2.1 mm, 2.4 μm, Thermo Scientific) and gradient elution on Waters Alliance 2695 system (Waters Corp.). The mobile phase consisted of methanol, water, and ammonium formate. Solvent A was 2 mM ammonium formate in methanol with 0.2% formic acid. The column was equilibrated with solvent A for 5 min. Samples were injected using the autosampler (an integral part of the Waters Alliance 2695 system) maintained at 10±2° C. The injection volumes were 80 μl for each sample. A complete injection of each sample took 7 min including column equilibration. The flow rate was 0.3 ml/min. The HPLC eluent was directly introduced to Quattro LC mass spectrometer (Micromass, Waters), equipped with an electrospray ion source that was used for ESI-MS/MS. The ESI-MS/MS experiments for the quantitation of sphingolipids were carried out in the positive ion mode with ESI needle voltage, 2.8 kV; source block temperature, 120° C.; desolvation temperature, 350° C.; desolvation gas flow, 540 liters/h; nebulizer gas flow, 80 liters/h; and collision gas pressure, 3.2×10⁻⁴ bars. Cone voltage and collision energy for each multiple reaction monitoring transition were optimized. Chromatographic data were analyzed by the QuanLynx module of the MassLynx software (Waters) to integrate the chromatograms for each multiple reaction monitoring transition.

Tumor Growth and Lung Colonization in Mice. All animal procedures were performed according to the National Institutes of Health and institutional guidelines, and were approved by the Wayne State University Animal Use and Care Committee. For subcutaneous implantation, lung carcinoma cells were adjusted to 1×10⁷ cells/ml. Mice were injected with 0.1 ml of cell suspension into the subcutaneous dorsa in the proximal midline. Alternatively, 1×10⁶ cells (in 50 μl) were injected via the tail vein route. NOD-Scid mice (8 weeks old, female, Taconic) were used for H1793, athymic nude mice (8 weeks old, female, Harlan) were used for H1299 cells, and C57BL/6 mice (8 weeks old, female, The Jackson Laboratory) were used for Lewis lung carcinoma cells. Tumor volume was measured in two dimensions using calipers, and volume was determined using the formula width 2×length×0.52 (49). For VPC23019 treatment, mice were randomized into two groups (six animals per group) 1 week after inoculation of tumor cells. One group of mice was intraperitoneally injected with VPC23019 (1.5 mg/kg of body weight), and the other was injected with 100 μl of 0.4% BSA (vehicle control) every 3 days. For TY-52156 treatment, mice (six mice) were intraperitoneally injected with TY-52156 (10 mg/kg of body weight) or DMSO control vehicle every 2 days.

Statistical Analysis. Results are shown as mean±S.D. Differences between paired samples were analyzed by Student's t test. ANOVA analysis was performed to analyze tumor progression in mouse experiments. p value <0.01 is considered highly significant, and p<0.05 is considered statistically significant.

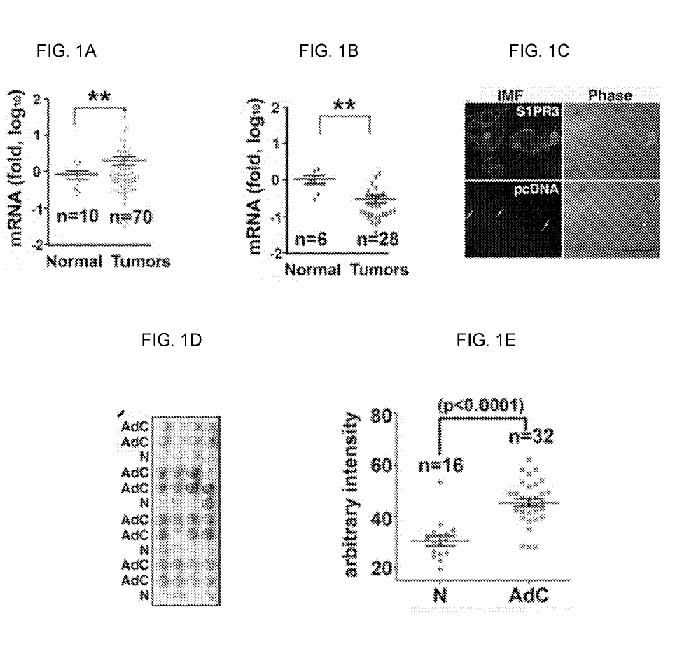

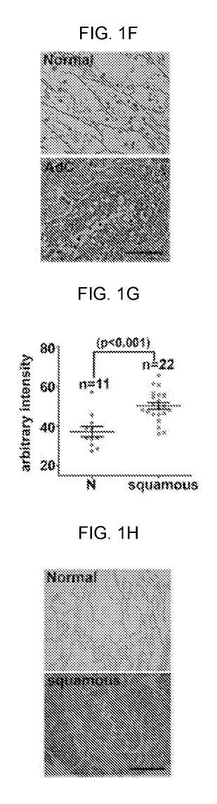

Results. Up-regulation of S1PR3 in Human Lung Adenocarcinomas. Previously, it was shown that levels of S1PR3 are significantly increased in a panel of cultured human lung adenocarcinoma cell lines when compared with normal lung epithelial cells (16). The pathological relevance of this in vitro observation was investigated by measuring mRNA levels of S1PR3 in cDNA microarrays of human lung adenocarcinoma specimens (Ori-Gene, HLRT). Quantitative PCR analysis showed that mRNA levels of S1PR3 are significantly increased in human lung adenocarcinoma specimens when compared with normal lung tissues (FIG. 1A). S1PR2 levels are increased in endothelial senescence and inflammation (10, 29). However, as shown herein, S1PR2 are decreased in human lung cancers (FIG. 1B).

Next, protein levels of S1PR3 examined by utilizing immunohistochemical staining in a paraffin-embedded tumor microarray of human lung adenocarcinoma specimens (Accumax). Anit-S1PR3 (Cayman) immunoreacted with plasma membrane-localized S1PR3 in HEK293 cells transiently transfected with S1PR3 vector. No immunoreactivity was observed in HEK293 transfected with pcDNA control vector (FIG. 1C).

Immunohistochemical staining analysis showed that the intensity of anti-S1PR3 immunostaining is significantly increased in human lung adenocarcinomas when compared with their respective adjacent normal lung epithelial cells (FIGS. 1D-1F). Moreover, levels of S1PR3 are increased in human lung squamous carcinoma specimens (FIGS. 1G and 1H).

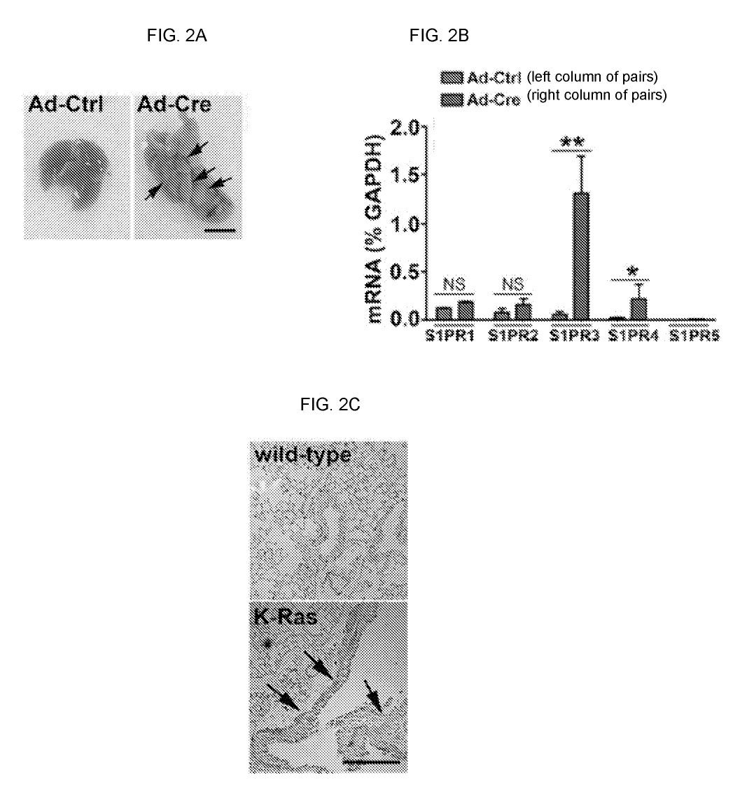

Oncogenic K-Ras mutation is found in more than 25% of non-small cell lung carcinomas and represents one of the most prevalent oncogenic drivers in non-small cell lung carcinomas (30, 31). A conditionally inducible knock-in K-Ras^{G12D} (Lox-Stop-Lox-K-Ras^{G12D}, LSL-K-Ras^{G12D}) mouse model (32, 33) was utilized to measure S1PR3 levels in lung adenocarcinomas and normal lung tissues. As shown in FIG. 2A, lung tumors were readily observed in heterozygous LSL-K-Ras^{G12D} mice following intratracheal injection of adenoviral particles carrying Cre recombinase (Ad-Cre). S1PR3 levels were increased 20-fold in lungs of K-Ras^{G12D}-expressing mice when compared with that in mice treated with empty adenoviral particles (Ad-Ctrl) (FIG. 2B). A minimal increase of S1PR4 was observed in lungs of K-Ras^{G12D}-expressing mice. There were no significant changes of S1PR1 and S1PR2, and S1PR5 was not detected in lungs of Ad-Cre-injected mice (FIG. 2B). In addition, immuno-histochemical staining showed that protein levels of S1PR3 were markedly increased in lung carcinoma specimens of K-Ras^{G12D} transgenic mice (FIG. 2C) when compared with normal lung tissues of wild-type mice. In a control, no staining was detected in lung adenocarcinoma specimens of K-Ras^{G12D} transgenic mice when immunohistochemical staining was performed without S1PR3 antibody (data not shown). These data suggest that S1PR3 levels are increased in lung adenocarcinomas.

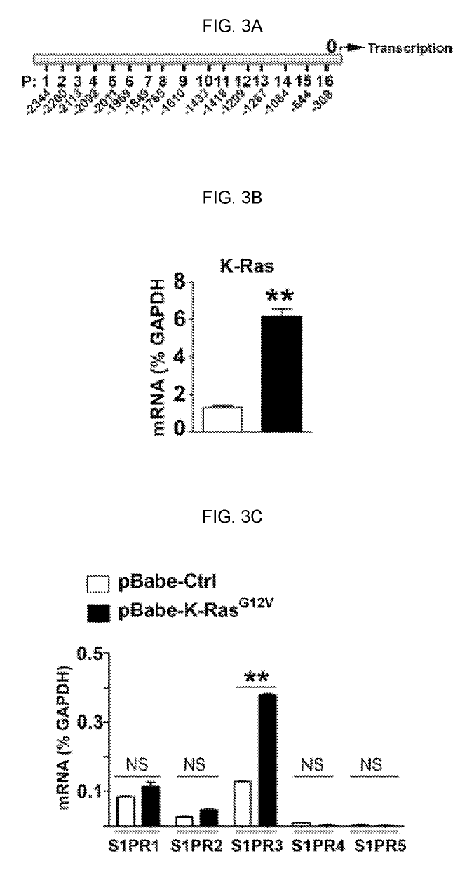

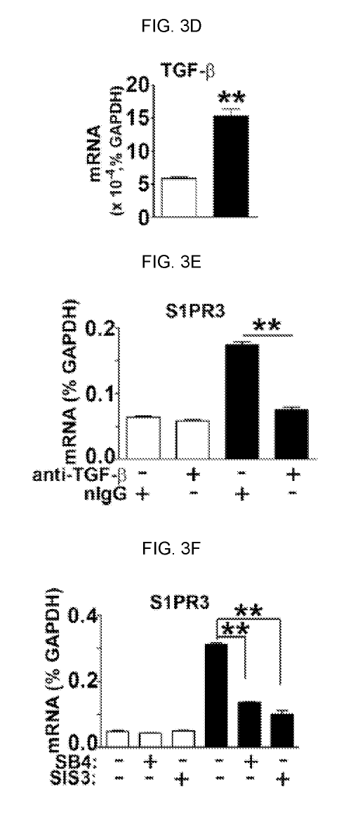

TGF-β/SMAD3 Signaling Pathway Stimulates S1PR3 Expression. Promoter analysis suggested that the promoter region of S1PR3 contains 16 potential binding elements for the SMAD3 molecule (FIG. 3A, FIG. 9), a critical signal transducer downstream of TGF-β/TGF-β receptor signaling. Also, it was shown that K-Ras mutant up-regulated TGF-β, which is required for tumor angiogenesis (34). Therefore, whether the TGF-β/SMAD3 signaling contributes to oncogenic K-Ras mutant-stimulated S1PR3 up-regulation was examined. Ectopic expression of oncogenic K-Ras^{G12V} mutant significantly increased S1PR3 (FIGS. 3B and 3C). Expression of K-Ras^{G12V} did not alter levels of other S1P receptor subtypes. In agreement with a previous study (34), levels of TGF-β were increased in K-Ras^{G12V}-expressing cells (FIG. 3D). Treatments with TGF-β antibody (FIG. 3E) and inhibition of TGF-β receptor I and SMAD3 using compound SB-431542 and SIS3 (FIG. 3F), respectively, abrogated the S1PR3 up-regulation in K-Ras^{G12V}-expressing cells.

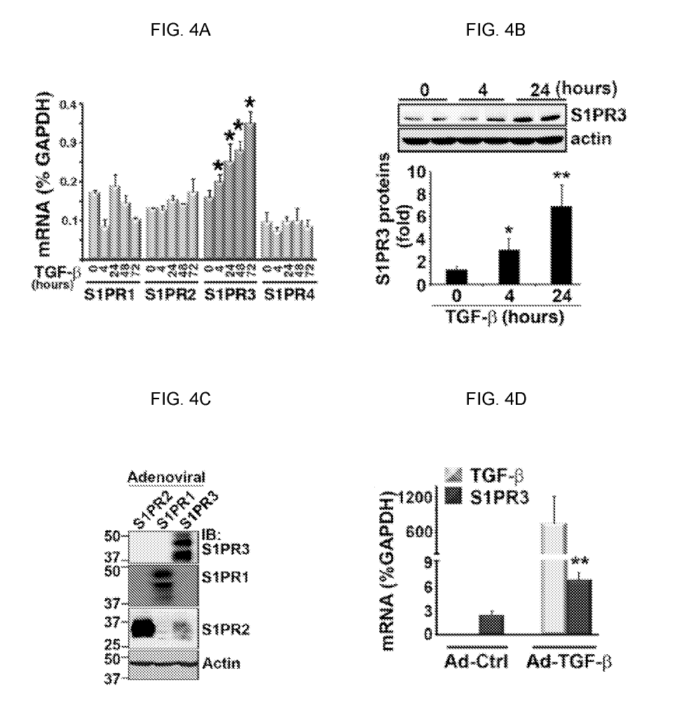

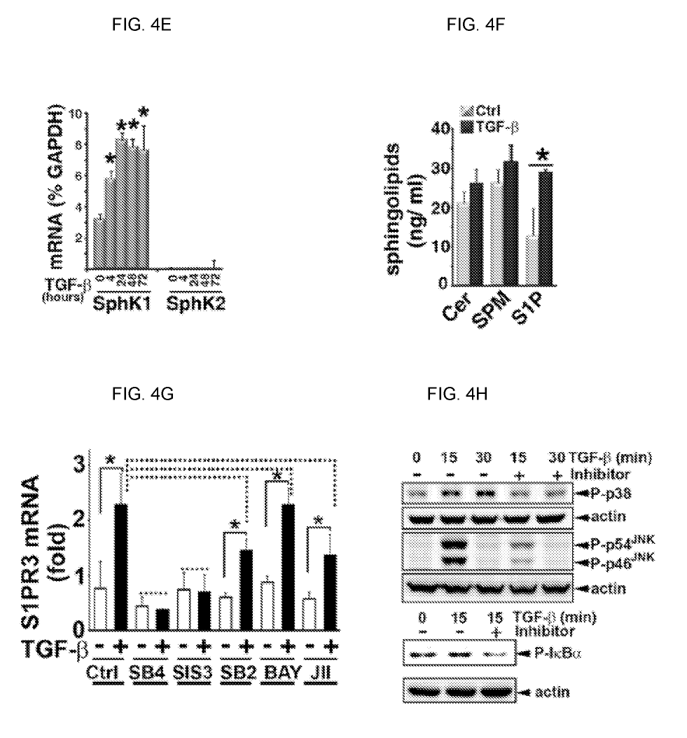

Next, experiments were conducted to investigate whether TGF-β treatment stimulates S1PR3 expression in lung epithelial cells. HBEC2-KT cells, an immortalized normal human lung epithelial cell line (16), were treated with TGF-β for various times. Quantitative analysis of the expression of S1P receptor subtypes by qPCR analysis showed that TGF-β treatment increased mRNA levels of S1PR3 in a time-dependent manner (FIG. 4A). TGF-β treatment did not affect levels of other subtypes of S1PRs such as S1PR1, S1PR2, and S1PR4. S1PR5 was not detected in HBEC2-KT cells. Also, TGF-β treatment increased protein levels of S1PR3 in HBEC2-KT cells (FIG. 4B). Validation of the specificity of anti-S1PR3 for Western blotting analysis showed that anti-S1PR3 specifically immunoreacts with S1PR3 (FIG. 4C).

Moreover, transduction with adenoviral particles carrying an active form of the TGF-β vector (35-37) effectively increased S1PR3 when compared with transduction with control adenoviral particles, in ex vivo mouse lung minces (FIG. 4D). Furthermore, TGF-β treatment time-dependently increased levels of SphK1 (FIG. 4E) and S1P production (FIG. 4F) in HBEC2-KT normal lung epithelial cells. TGF-β treatment did not alter levels of SphK2 in HBEC2-KT cells.

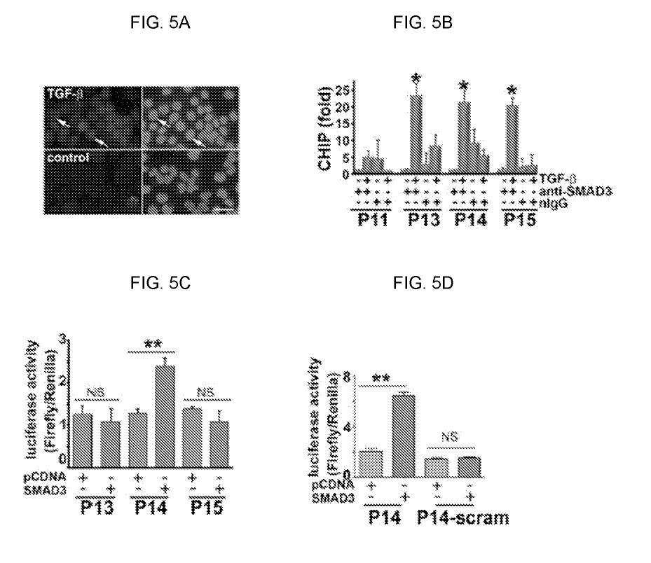

Next, a selective pharmacological inhibitor was used to investigate the role of SMAD3 in TGF-β-stimulated S1PR3 up-regulation. Inhibition of TGF-β receptor I and SMAD3 using compound SB-431542 and SIS3, respectively, abrogated the TGF-β-stimulated S1PR3 up-regulation (FIG. 4G). In contrast, inhibition of other signaling molecules downstream of TGF-β signaling (e.g. NFκB, JNK, and p38 kinase) did not significantly diminish the TGF-β-stimulated S1PR3 up-regulation. In a parallel control experiment, treatment with inhibitor effectively diminished the activation of their respective target following TGF-β stimulation (FIG. 4H). These data suggest that the TGF-β receptor I/Samd3 signaling pathway contributes to the TGF-β-stimulated S1PR3 expression in lung epithelial cells. Treatment of HBEC2-KT cells with TGF-β markedly stimulated the nuclear accumulation of phosphorylated SMAD3 (FIG. 5A, arrows), indicating that TGF-β treatment activates SMAD3 in HBEC2-KT lung epithelial cells. Subsequently, 16 pairs of primers (FIG. 9) were designed that amplify these candidate SMAD3 binding elements in the promoter region of S1PR3. ChIP assay showed that TGF-β treatment significantly increased the binding of phospho-SMAD3 to P13, P14, and P15 sites in the promoter region of S1PR3 (FIG. 5B). No specific binding was observed when ChIP assays were performed using irrelevant normal IgG as a control, suggesting that bindings of phospho-SMAD3 are specific.

Next, a luciferase reporter assay was used to examine whether SMAD3 transactivates those candidate SMAD3 binding sites present in the S1PR3 promoter region. As shown in FIG. 5C, SMAD3 activates PGL3-promoter luciferase vector carrying P14, whereas SMAD3 did not activate PGL3-promoter luciferase vector carrying P13 and P15. The luciferase reporter assay is specific, because SMAD3 was unable to activate scrambled P14 (FIG. 5D).

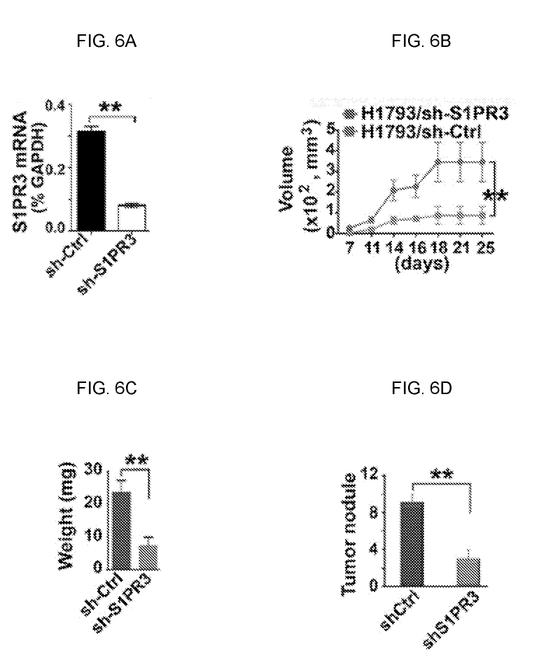

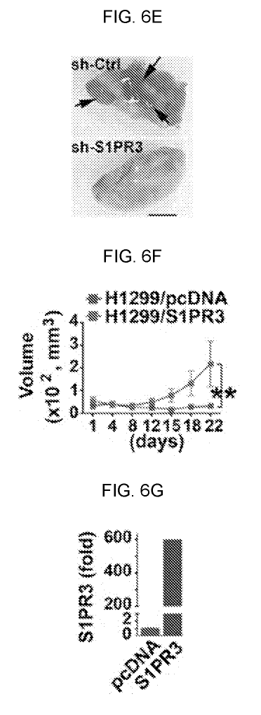

S1PR3 Promotes Lung Adenocarcinoma Progression. It was previously shown that S1PR3 activation promotes proliferation, soft agar growth, and invasion of human lung adenocarcinoma cells in vitro (11, 16). Therefore, animal models were utilized to examine the role of S1PR3 in human lung adenocarcinoma progression. Human H1793 lung adenocarcinoma cells, abundantly expressing S1PR3 (16), were stably transfected with sh-S1PR3 or sh-control vectors. Expression of sh-S1PR3 effectively knocked down 67% of S1PR3 in H1793 cells (FIG. 6A). Moreover, S1PR3 knockdown significantly inhibited tumor growth in a subcutaneous xenograft mouse model (FIGS. 6B and 6C). Similarly, S1PR3 knockdown diminished lung colonization of H1793 cells, which were injected via the tail vein route (FIGS. 6D and 6E). In contrast, H1299 human lung adenocarcinoma cells express very low levels of S1PR3 among human lung adenocarcinoma cell lines (16) and are poorly tumorigenic in athymic mice. Ectopic expression of S1PR3 profoundly promoted tumor growth in athymic mice (FIG. 6F). These results suggest that S1PR3 activity promotes tumorigenesis of human lung adenocarcinomas.

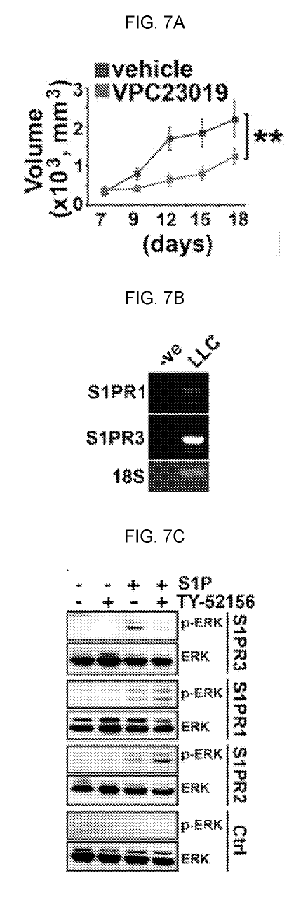

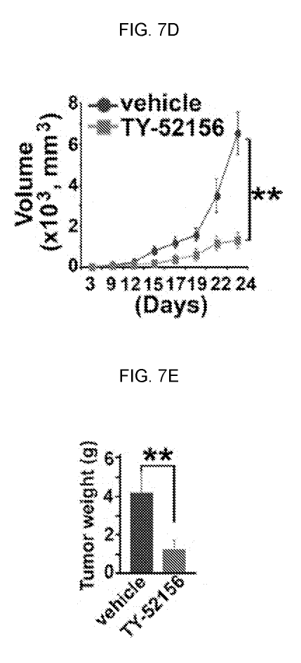

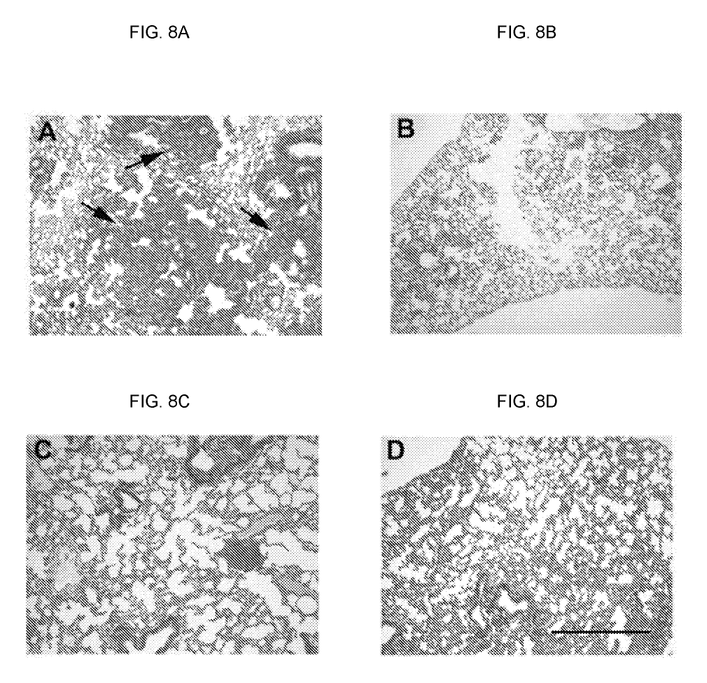

Pharmacological Inhibition of S1PR3 Diminishes Lung Adenocarcinoma Growth. Next, experiments were conducted to investigate whether treatment with S1PR3 antagonist diminishes the growth of human lung adenocarcinoma cells. C57BL/6 mice were subcutaneously implanted with murine Lewis lung carcinoma (LLC) cells. 1 week after tumor implantation, mice were intraperitoneally injected every 3 days with VPC23019, an antagonist of S1PR1 and S1PR3 receptors (38). Administration of VPC23019 significantly inhibited tumor growth (FIG. 7A). Lewis lung carcinoma cells predominantly express S1PR3, and S1PR1 is barely detected (FIG. 7B). Thus, the effect of VPC23019 on inhibition of tumor growth is most likely due to its antagonistic activity on S1PR3 present in LLC cells. Indeed, treatment with TY-52156, a highly selective antagonist of S1PR3 (39-41) (FIG. 7C), significantly suppressed the growth of Lewis lung carcinoma cells (FIGS. 7D and 7E). These results suggest that S1PR3 represents a novel therapeutic target for the treatment of lung carcinomas.

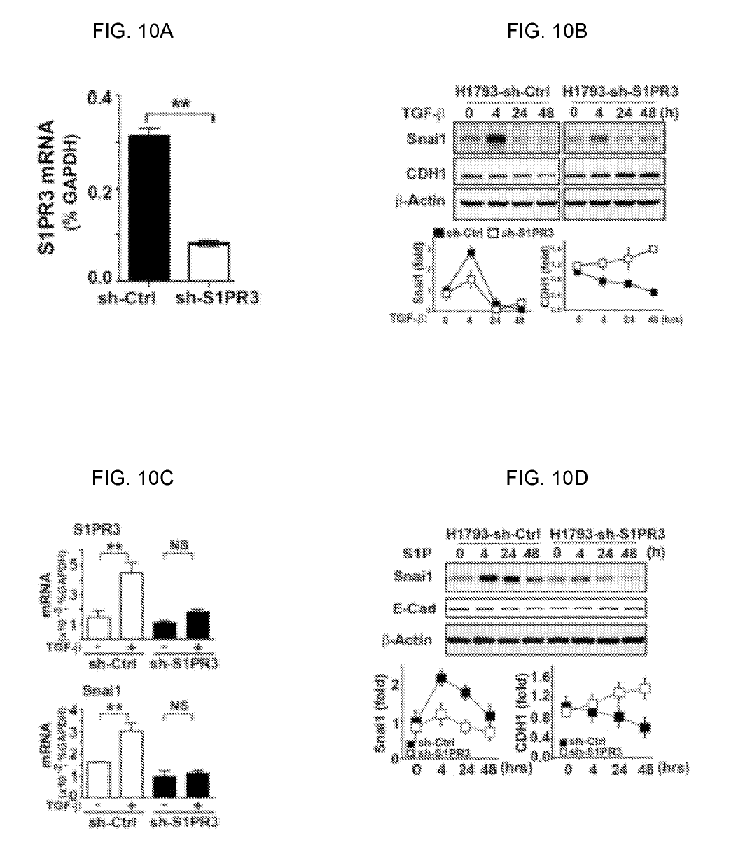

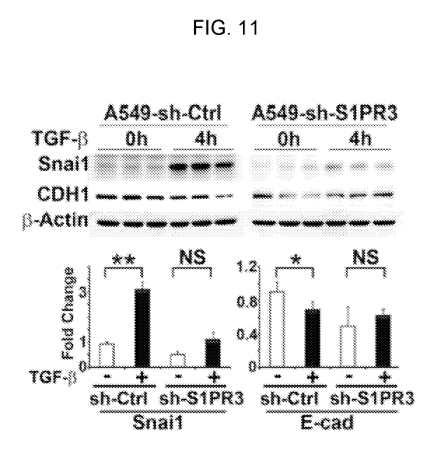

S1PR3 regulates the TGF-β-mediated Snai1/E-cadherin signaling pathway. TGF-β regulates the Snai1/E-cadherin (CDH1) signaling pathway that promotes cancer progression. Therefore, whether S1PR3 regulates the TGF-β-mediated Snai1 up-regulation and E-cadherin suppression was examined. Human H1793 lung adenocarcinoma cells, abundantly expressing S1PR3 (16), were stably transfected with sh-S1PR3 or sh-control vectors. Expression of sh-S1PR3 effectively knocked-down 67% of S1PR3 in H1793 cells (FIG. 10A). TGF-β treatment markedly increased protein levels of Snai1 at 4 hours after treatment, and time-dependently decreased E-cadherin proteins in H1793 cells transfected with sh-control vector (FIG. 10B). In contrast, the TGF-β-stimulated Snai1 increase was diminished, and E-cadherin suppression was abrogated in H1793 cells transfected with sh-S1PR3 vector. Similar results were obtained in A549 lung adenocarcinoma cells (FIG. 11). Furthermore, TGF-β treatment significantly increased mRNA levels of S1PR3 and Snai1 in HBEC2-KT lung epithelial cells. S1PR3 knockdown abrogates the TGF-β-stimulated Snai1 up-regulation (FIG. 10C). In addition, S1P treatment time-dependently increased Snai1 and suppressed E-cadherin in H1793 cells transfected with sh-control vector, whereas the Snai1 increase and E-cadherin suppression was profoundly diminished in H1793 cells transfected with sh-S1PR3 (FIG. 10D). These results suggest that S1P/S1PR3 signaling regulates the TGF-β mediated Snai1/E-cadherin signaling axis.

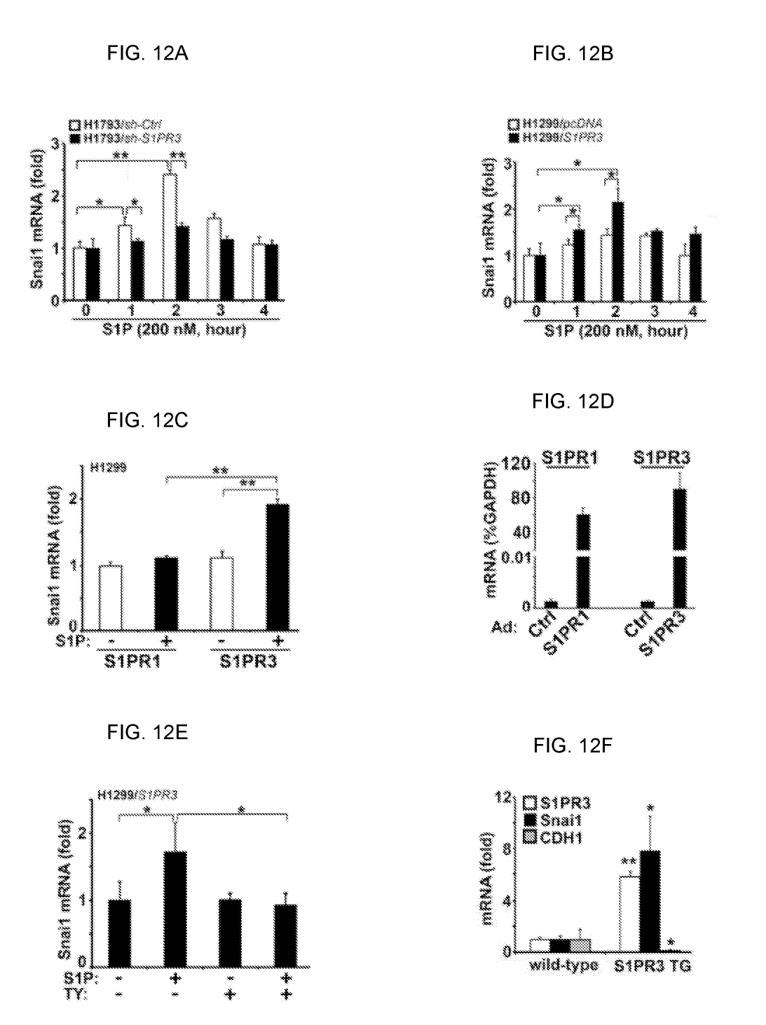

Molecular approaches and pharmacological reagents were next used to further examine the role of S1PR3 in stimulating the Snai1 expression in human lung adenocarcinoma cells. Up-regulation of Snai1 mRNA is readily observed at 1 hour, and peaked at 2 hours after S1P treatment in H1793 cells (FIG. 12A). The S1P-stimulated Snai1 expression was abrogated in H1793 cells knocked-down of S1PR3. S1PR3 is barely detected in H1299 human lung adenocarcinoma cells (16). S1P treatment was unable to induce Snai1 expression in H1299 cells (FIG. 12B). However, S1P treatment time-dependently increased Snai1 after ectopic expression of S1PR3 in H1299 cells. Moreover, H1299 cells were transduced with adenoviral particles carrying S1PR1 or S1PR3 vector. H1299 expressing S1PR3 cells, and not H1299 expressing S1PR1 cells, increased Snai1 expression following S1P treatment (FIGS. 12C, 12D), suggesting the specificity of S1PR3 in stimulating Snai1 expression. TY-52156 (TY, >99% purity), a small molecule shown to inhibit S1PR3 activity (39-41) was chemically synthesized. Treatment with TY-52156 inhibited S1P/S1PR3, and had no effect on S1P/S1PR1 and S1P/S1PR2-stimulated ERK1/2 activation (data not shown), indicating that TY-52156 is a highly selective antagonist of S1PR3. TY-52156 treatment completely abrogated the S1P-increased Snai1 expression in H1299 cells expressing S1PR3 (FIG. 12E). Furthermore, a transgenic mouse strain in which S1PR3 is specifically overexpressed in lung tissues (S1PR3lung/lung) was recently generated by using the surfactant protein C driven expression vector (data now shown). Snai1 levels are markedly increased and E-cadherin levels are reduced in lung tissues of S1PR3lung/lung transgenic mice (FIG. 12F). The data suggest that S1P/S1PR3 signaling stimulates Snai1 transcription, leading to E-cadherin suppression in lung epithelial cells.

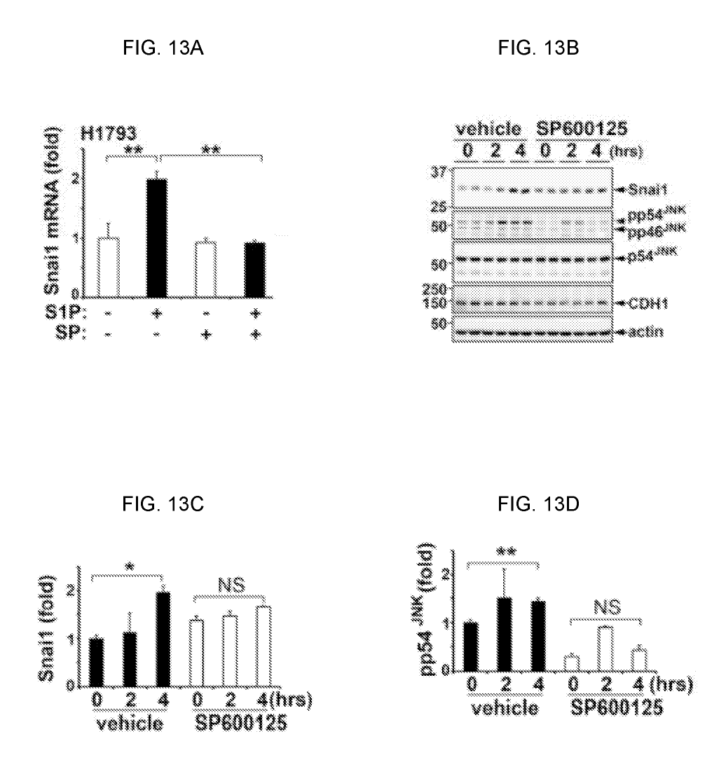

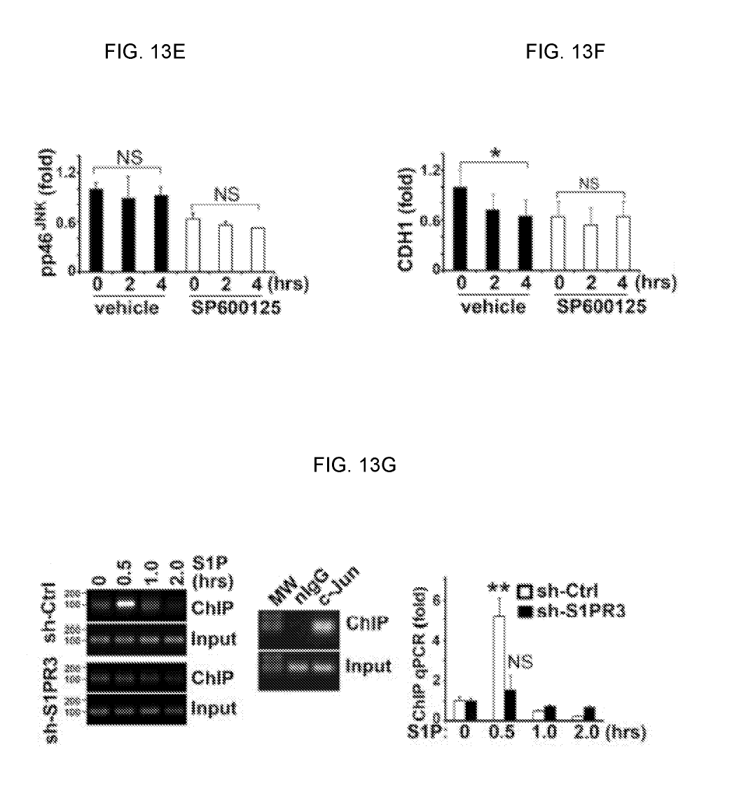

S1P/S1PR3 signaling stimulates Snai1 expression via the JNK/AP-1 signaling pathway. Promoter analysis suggested that there are several candidate AP-1 binding sites on the promoter region of Snai1. S1P/S1PR3 activates the JNK/AP-1 signaling pathway in human lung adenocarcinoma cells (11). Therefore, whether S1P/S1PR3 signaling stimulates Snai1 expression mediated by the JNK/Snai1 pathway was investigated. pp54JNK was activated up to 4 hours following S1P treatment in human lung adenocarcinoma cells (FIGS. 13B, 13C). Treatment with JNK inhibitor diminished the S1P-stimulated pp54JNK activation. Moreover, treatment with JNK inhibitor abrogated the SiP-induced Snai1 expression at mRNA (FIG. 13C) and protein levels (FIGS. 13B, 13C), and E-cadherin suppression at protein level (FIGS. 13B, 13F). Chromatin immunoprecipitation assay showed that S1P treatment stimulated the binding of AP-1 to the promoter region of Snai1 in H1793 human lung adenocarcinoma cells. In contrast, S1P was unable to stimulate AP-1 binding to Snai1 promoter in H1793 cells which S1PR3 were knocked-down (FIG. 13G). There was no binding detected when chromatin immunoprecipitation assay was performed with irrelevant normal IgG (middle panel, FIG. 13G), indicating that the chromatin immunoprecipitation assay is specific. These data suggest that S1P/S1PR3 signaling regulates Snai1/E-cadherin pathway via the JNK/AP-1 pathway.

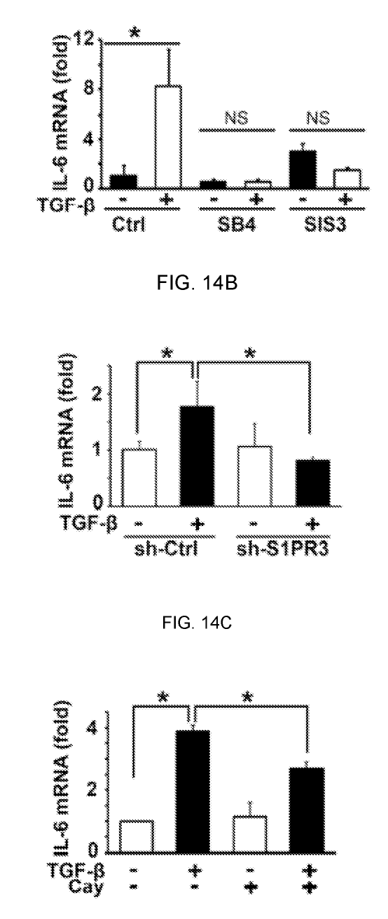

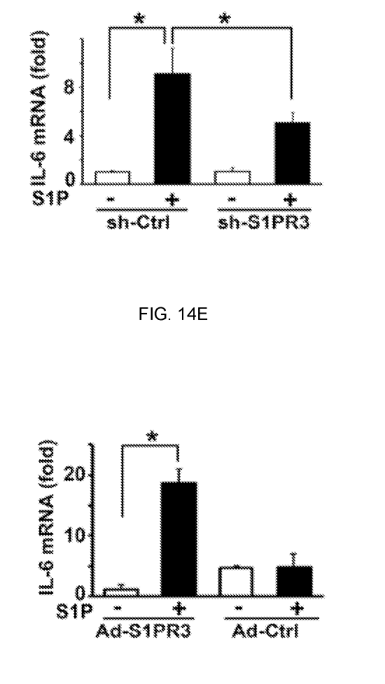

TGF-β plays an important role in regulating the tumorigenic processes including epithelial-mesenchymal transition (EMT)(17, 20, 64-66) and tumor inflammation (67-70). The TGF-β-mediated Snai1/E-cadherin pathway has a critical role in EMT. S1PR3 knockdown attenuated the TGF-β-mediated Snai1/E-cadherin pathway. Moreover, it was shown for the first time that S1P is capable of activating the Snai1/E-cadherin pathway, which is dependent on S1PR3. Mechanistically, it was demonstrated that the S1PR3-mediated JNK/AP-1 pathway contributes to Snai1 up-regulation and E-cadherin suppression. Furthermore, TGF-β stimulates tumor inflammation. For example, TGF-β stimulates the expression of pro-inflammatory and pro-tumorigenic cytokine IL-6 (71, 72). Elevated systemic and pulmonary productions of IL6 are commonly observed in lung adenocarcinoma patients and correlate with poor patient survival (73, 74). The TGF-β/IL-6 axis was recently shown to mediate the chemo-resistance in lung cancer (71). The TGF-β-induced IL-6 production is mediated by the TGF-β receptor and Smad3 pathway in human lung adenocarcinoma cells (FIG. 14A). Thus, whether S1PR3 regulates the TGF-β-stimulated IL-6 production was examined. S1PR3 knockdown (FIG. 14B) or inhibition (FIG. 14C) significantly diminished the TGF-β-stimulated IL-6 production in lung adenocarcinoma cells. Also, S1P treatment increased IL-6 production which is dependent on S1PR3 (FIG. 14D). Levels of IL-6 were significantly increased in HBEC2-KT normal lung epithelial cells ectopically expressing S1PR3, following S1P stimulation (FIG. 14E).

Discussion Levels of S1PR3 are increased in a panel of cultured human lung adenocarcinoma cell lines when compared with normal lung epithelial cells (16). In this Example it was shown that mRNA and protein levels of S1PR3 are significantly up-regulated in human lung adenocarcinoma specimens. This observation is supported by the analysis of Oncomine data sets (42-45) showing that S1PR3 expression correlates with clinical stages (42, 44), EML4-ALK gene fusion (42), lymphatic and perineural invasion (44), metastasis to bone (44), vascular invasion (44), BCL amplification (45), and APC deletion and family history (43) of human lung adenocarcinomas. These data shown that S1PR3 is up-regulated in human lung adenocarcinomas, and S1PR3 expression correlates with the aggressiveness of lung adenocarcinomas.

Oncogenic K-Ras mutation is found in more than 25% of non-small cell lung cancers (30, 31). In the LSL-K-Ras^{G12D} transgenic mouse model, the expression of K-Ras^{G12D} mutant triggered the development of lung cancers and concurrently stimulated the expression of S1PR3. In agreement with this study, Oncomine data sets analysis showed that S1PR3 up-regulation correlates with K-Ras mutation status in human lung cancers (42, 43, 48, 50, 51) (see Genomic Data Commons). Mechanistically, the data suggest that the oncogenic K-Ras mutant-stimulated S1PR3 expression is mediated by an autocrine TGF-β/SMAD3 axis in lung epithelial cells. In supporting these observations, it was shown that oncogenic K-Ras mutant stimulated the expression of TGF-β, which plays a critical role in tumor angiogenesis in K-Ras mutant-driven cancers (34). It should be noted that lung cancers driven by K-Ras mutant are generally refractory to chemotherapy as well as targeted agents (31, 52). To date, the identification of drugs to therapeutically inhibit K-Ras mutant has been unsuccessful, suggesting that other approaches are required. The present Example shows that oncogenic K-Ras mutant stimulates S1PR3 expression, showing that S1PR3 represents a novel therapeutic target for the treatment of K-Ras mutant-driven lung cancers.

S1PR3 regulates the proliferation, colony formation, and invasiveness of human lung adenocarcinoma cells in vitro (11, 16). In the present Example, animal models were utilized to examine the role of S1PR3 in the progression of human lung adenocarcinomas. H1793 human lung adenocarcinoma cells abundantly express S1PR3, and S1PR3 knockdown profoundly abrogated proliferation, colony formation in soft agar, and invasion of tumor cells in vitro (11, 16). Similarly, S1PR3 knockdown significantly inhibited tumor growth in a xenograft model, as well as lung colonization of adenocarcinoma cells in a tail vein implantation model. In contrast, H1299 human lung adenocarcinoma cells express very low levels of S1PR3 among lung adenocarcinoma cell lines (16). Expression of S1PR3 significantly promoted growth of tumor xenograft. These results suggest that the S1PR3-mediated signaling pathways play an important role in promoting the progression of lung adenocarcinoma cells. Two S1PR3-mediated signaling pathways have been characterized that may have functional implications in promoting lung adenocarcinoma progression. It was found that S1PR3 activation transcriptionally up-regulates EGFR levels and greatly potentiates the effect of EGF on the proliferation of lung adenocarcinoma cells (16). Moreover, a novel signaling pathway was characterized, namely S1PR3/JNK/AP-1/ETS-1/CD44 axis, which critically regulates the invasiveness of human lung adenocarcinoma cell in vitro (11). Collectively, the Example data suggests that S1PR3 represents a therapeutic target for the treatment of human lung adenocarcinomas. Indeed, the experiment using pharmacological inhibitors supports this notion. Administration of VPC23019 (an antagonist of S1PR1 and S1PR3 receptors (38)) and TY-52156 (a selective inhibitor of S1PR3 (39-41)) significantly diminished lung tumor growth in xenograft mouse model.

Mechanistically, it was shown that TGF-β/SMAD3 signaling pathway transactivates S1P/S1PR3 axis in lung epithelial cells. A previous study showed that TGF-β activates sphingosine kinase via a non-SMAD signaling pathway and that the TGF-β sphingosine kinase axis is important for the migration and invasion of esophageal cancer cells in vitro (53). However, the role of the TGF-β signaling axis on the regulation of S1PRs was not investigated in that study. Moreover, in agreement with the observations described herein, Cencetti et al. (54) showed that TGF-β stimulated S1PR3 expression in C2C12 myoblasts. In contrast to their study, the present Example precisely defined the SMAD3 binding sites on the promoter region of S1PR3 and demonstrated that the TGF-β stimulated S1PR3 up-regulation is dependent on the SMAD3 signaling molecule. Furthermore, it was found that TGF-β concomitantly stimulated SphK1 expression and increased S1P production in lung epithelial cells. Collectively, the results show that TGF-β activates an autocrine S1P/S1PR3 signaling in lung epithelial cells, which contributes to lung adenocarcinoma progression.

Several tumors, including lung cancers, express high levels of TGF-β (55-57), which correlates with tumor progression and clinical prognosis (58-63). Thus, the observation of TGF-β-mediated S1PR3 up-regulation in lung cancers is pathologically relevant. In addition, TGF-β plays an important role in regulating the tumorigenic processes including epithelial-mesenchymal transition (17, 20, 64-66) and tumor inflammation (67-72). For example, TGF-β stimulates the expression of pro-inflammatory and pro-tumorigenic cytokine IL-6 (71, 72). Elevated systemic and pulmonary productions of IL-6 are commonly observed in lung adenocarcinoma patients and correlate with poor patient survival (73, 74). Moreover, the TGF-β/IL-6 axis was recently shown to mediate the chemo-resistance in lung cancer (71). The results described here show that TGF-β activates the autocrine S1P/S1PR3 signaling axis in lung epithelial cells.

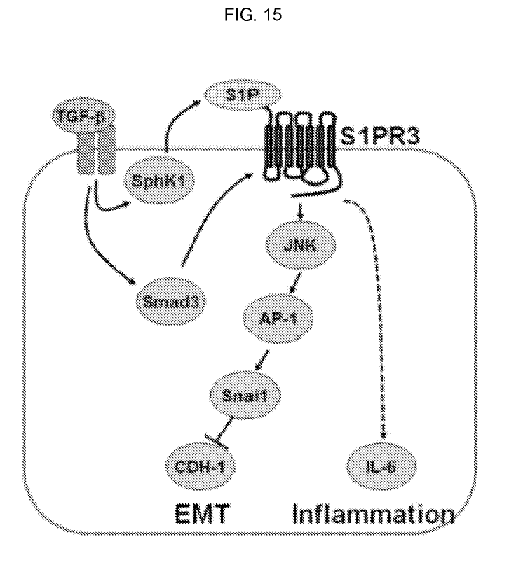

A model consistent with the current data and disclosure is depicted in FIG. 15.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, a material effect is a statistically signification reduction in the ability of a TY-52156 compound to inhibit lung colonization of adenocarcinoma cells in a tail vein implantation method.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

### NUMBERED REFERENCES

1. Siow, et al., (2011) Adv. Enzyme Regul. 51, 229-244
2. Lee, et al., (1998) Science 279, 1552-1555
3. Hait, et al., (2009) Science 325, 1254-1257
4. Alvarez, et al., (2010) Nature 465, 1084-1088
5. Lee, et al., (1999) Cell 99, 301-312
6. Green et al., (2011) Nat. Immunol. 12, 672-680
7. Jenne, et al., (2009) J. Exp. Med. 206, 2469-2481
8. Lee, et al., (2001) Mol. Cell 8, 693-704
9. An, et al., (1998) J. Cell. Biochem. Suppl. 30-31, 147-157
10. Estrada, et al., (2008) J. Biol. Chem. 283, 30363-30375
11. Zhang, et al., (2013) J. Biol. Chem. 288, 32126-32137
12. Yester, et al., (2011) Cancer Metastasis Rev. 30, 577-597
13. Pyne, et al., (2012) Biochem. Soc. Trans. 40, 94-100
14. Furuya, et al., (2011) Cancer Metastasis Rev. 30, 567-576
15. Aoyagi, et al., (2012) Lymphat. Res. Biol. 10, 97-106
16. Hsu, et al., (2012) Int. J. Oncol. 40, 1619-1626
17. Papageorgis, (2015) J. Oncol. 2015, 587193
18. Nowrin, et al., (2014) Expert Rev. Respir. Med. 8, 547-559
19. Giannelli, et al., (2014) Cancer Res. 74, 1890-1894
20. Derynck, et al., (2014) Curr. Opin Cell Biol. 31, 56-66
21. Fuxe & Karlsson, (2012) Semin. Cancer Biol. 22, 455-461
22. Nalluri, et al., (2015) Cytoskeleton (Hoboken) 72, 557-569
23. Mantel & Schmidt-Weber, (2011) Methods Mol. Biol. 677, 303-338
24. Nagaraj & Datta, (2010) Expert Opin. Investig. Drugs 19, 77-91
25. Derynck, et al., (2001) Nat. Genet. 29, 117-129
26. Yamanaka, et al., (2004) J. Biol. Chem. 279, 53994-54001
27. Xin, et al., (2004) J. Biol. Chem. 279, 35255-35262
28. Bu, et al., (2008) J. Biol. Chem. 283, 19593-19602
29. Zhang, et al., (2013) Prostaglandins Other Lipid Mediat. 106, 62-71
30. Sholl, et al., (2015) J. Thorac. Oncol. 10, 768-777
31. Karachaliou, et al., (2013) Clin. Lung Cancer 14, 205-214
32. Jackson, et al., (2001) Genes Dev. 15, 3243-3248
33. Zhao, et al., (2013). Oncogene 32, 5186-5190
34. Rak, et al., (1995) Cancer Metastasis Rev. 14, 263-277
35. Gauldie & Kolb, (2008) Am. J. Physiol. Lung Cell. Mol. Physiol. 294, L151
36. Decologne, et al., (2007) J. Immunol. 179, 6043-6051
37. Cui, et al., (2011) Int. J. Biochem. Cell Biol. 43, 1122-1133
38. Davis, et al., (2005) J. Biol. Chem. 280, 9833-9841
39. Murakami, et al., (2010) Mol. Pharmacol. 77, 704-713
40. Nussbaum et al., (2015) Nat. Commun. 6, 6416
41. Hirata, et al., (2014) Nat. Commun 5, 4806
42. Bild, et al., (2006) Nature 439, 353-357
43. Ding et al., (2008) Nature 455, 1069-1075
44. Larsen, et al., (2007) Carcinogenesis 28, 760-766
45. Olejniczak, et al., (2007) Mol. Cancer Res. 5, 331-339
46. Zhang, et al., (2014) J. Biol. Chem. 289, 32178-32185
47. Xie, et al., (2011) J. Biol. Chem. 286, 15050-15057
48. Okayama, et al., (2012) Cancer Res. 72, 100-111
49. Yamamoto, et al., (2009) Lung Cancer 63, 315-321
50. Selamat, et al., (2012) Genome Res. 22, 1197-1211
51. Yauch, et al., (2005) Clin. Cancer Res. 11, 8686-8698
52. Riely, et al., (2009) Proc. Am. Thorac. Soc. 6, 201-205
53. Miller, et al., (2008) Mol. Cell. Biol. 28, 4142-4151
54. Cencetti, et al., (2010) Mol. Biol. Cell 21, 1111-1124
55. Barthelemy-Brichant, et al., (2002) Eur. J. Clin. Invest. 32, 193-198
56. Domagala-Kulawik, et al., (2006) Arch. Immunol. Ther. Exp. (Warsz.) 54, 143-147
57. Lee, et al., (2004) J. Immunol. 172, 7335-7340
58. Hasegawa, et al., (2001) Cancer 91, 964-971
59. Bruna, et al., (2007) Cancer Cell 11, 147-160
60. Saito, et al., (1999) Cancer 86, 1455-1462
61. Tsushima, et al., (1996) Gastroenterology 110, 375-382
62. Wikstrom et al., (1998) Prostate 37, 19-29
63. Levy & Hill, (2006) Cytokine Growth Factor Rev. 17, 41-58
64. Yu, et al., (2015) Neoplasma 62, 1-15
65. Wang, et al., (2013) Curr. Cancer Drug Targets 13, 963-972
66. Saitoh, (2015) Cancer Sci. 106, 481-488
67. Yang, (2010) Cancer Metastasis Rev. 29, 263-271
68. Tian, et al., (2011) Cell. Signal. 23, 951-962
69. Naber, et al., (2008) Curr. Cancer Drug Targets 8, 466-472
70. Hong, et al., (2010) World J. Gastroenterol. 16, 2080-2093
71. Yao, et al., (2010) Proc. Natl. Acad. Sci. U.S.A. 107, 15535-15540
72. Chenn et al., (2012) Clin. Sci. (Lond.) 122, 459-472
73. Yanagawa, et al., (1995) Br. J. Cancer 71, 1095-1098
74. Haura, et al., (2006) Clin. Lung Cancer 7, 273-275
75. Ivanova, et al., (2009) Mol. Cell. Endocrinol. 305, 12-21

What is claimed is:

1. A method of treating non-small cell lung cancer (NSCLC) in a subject in need thereof comprising administering a therapeutically effective amount of the TY-52156 compound 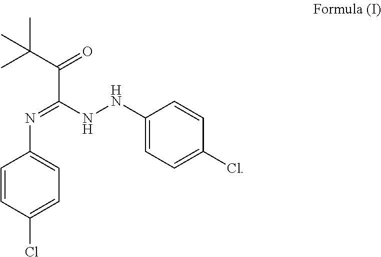 [SMILES: CC(C)(C)C(=O)/C(=N/c1ccc(Cl)cc1)NNc1ccc(Cl)cc1]
to the subject, thereby treating the NSCLC in the subject.

2. A method of treating cancer in a subject in need thereof comprising administering a therapeutically effective amount of a TY-52156 compound to the subject, thereby treating the cancer in the subject.

3. The method of claim 2, wherein the TY-52156 compound has the following structural formula:

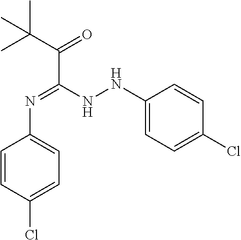 [SMILES: CC(C)(C)C(=O)/C(=N/c1ccc(Cl)cc1)NNc1ccc(Cl)cc1]

4. The method of claim 2, wherein the cancer is mediated by a KRAS mutation and/or TGF-β/Smad3 signaling.

5. The method of claim 2, wherein the cancer is lung cancer, breast cancer, colon cancer, pancreatic cancer, bladder cancer, ovarian cancer, prostate cancer, or leukemia.

6. The method of claim 4, wherein the cancer mediated by a KRAS mutation is lung cancer, colon cancer, pancreatic cancer, or leukemia.

7. The method of claim 5, wherein the lung cancer is non-small cell lung cancer (NSCLC).

8. A method for determining whether therapy comprising a TY-52156 compound is appropriate for a subject diagnosed with cancer, wherein the method comprises obtaining a biological sample from the subject; testing the biological sample for the presence of a KRAS gene mutation and/or evidence of TGF-β/Smad3 signaling; and determining that therapy with a TY-52156 compound is appropriate for the subject if the KRAS gene mutation and/or evidence of TGF-β3/Smad3 signaling is present.

9. The method of claim 8, wherein the evidence of TGF-β3/Smad3 signaling includes increased S1PR3, increased SphK1, increased S1P, increased Snai1, increased interleukin 6, or decreased E-cadherin.

10. The method of claim 8, wherein the method further comprises confirming therapy with a TY-52156 compound is appropriate by culturing cells of the biological sample and administering a TY-52156 compound to the cultured cells wherein the TY-52156 compound inhibits cell growth in the culture.

11. The method of claim 8, wherein the biological sample is a tissue biopsy sample, a tumor biopsy sample, a blood sample, a serum sample, a saliva sample, a urine sample, or a bronchoalveolar larvage sample.

12. The method of claim 8, wherein the cancer is lung cancer, colon cancer, pancreatic cancer, or leukemia.

13. The method of claim 12, wherein the cancer is lung cancer.

14. The method of claim 13, wherein the lung cancer is NSCLC.

15. The method of claim 8, wherein the TY-52156 has the following structural formula: 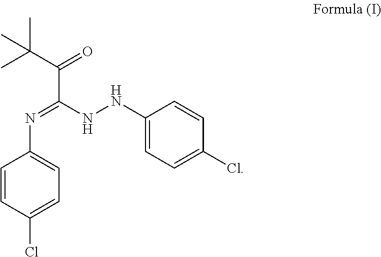 [SMILES: CC(C)(C)C(=O)/C(=N/c1ccc(Cl)cc1)NNc1ccc(Cl)cc1]


SEQUENCE LISTING (57 sequences)

SEQ ID NO: 1 - protein, 189 aa, Homo sapiens [sequence omitted]

SEQ ID NO: 2 - DNA, 22 bp, Artificial Sequence
  human and mouse S1PR1 Sense
atcatgggctggaactgcatca

SEQ ID NO: 3 - DNA, 23 bp, Artificial Sequence
  human and mouse S1PR1 Antiense
cgagtcctgaccaaggagtagat

SEQ ID NO: 4 - DNA, 22 bp, Artificial Sequence
  human and mouse S1PR2 sense
cagacgctagccctgctcaaga

SEQ ID NO: 5 - DNA, 19 bp, Artificial Sequence
  human and mouse S1PR2 antisense
tagtgggctttgtagagga

SEQ ID NO: 6 - DNA, 22 bp, Artificial Sequence
  human and mouse S1PR3 sense
acaaccgcatgtactttttcat

SEQ ID NO: 7 - DNA, 22 bp, Artificial Sequence
  human and mouse S1PR3 antisense
tactgccctccctgaggaacca

SEQ ID NO: 8 - DNA, 20 bp, Artificial Sequence
  human S1PR4, sense
gggccatcttccgcctggtg

SEQ ID NO: 9 - DNA, 20 bp, Artificial Sequence
  human S1PR4, antisense
tgccccgcaggtactcctgg

SEQ ID NO: 10 - DNA, 20 bp, Artificial Sequence
  human S1PR5, sense
ggcgcgcacctgtcctgtac

SEQ ID NO: 11 - DNA, 20 bp, Artificial Sequence
  human S1PR5, antisense
tcgggtctctgccgcaggag

SEQ ID NO: 12 - DNA, 20 bp, Artificial Sequence
  human and mouse SphK1, sense
aaacccctgtgtagcctccc

SEQ ID NO: 13 - DNA, 20 bp, Artificial Sequence
  human and mouse SphK1, antisense
agcaggttcatgggtgacag

SEQ ID NO: 14 - DNA, 18 bp, Artificial Sequence
  human SphK2, sense
gcacagcaacagtgagca

SEQ ID NO: 15 - DNA, 18 bp, Artificial Sequence
  human SphK2, antisense
gagcctgagtgagtggga

SEQ ID NO: 16 - DNA, 21 bp, Artificial Sequence
  porcine TGF-Beta, sense
gcacgtggagctataccagaa

SEQ ID NO: 17 - DNA, 20 bp, Artificial Sequence
  porcine TGF-Beta, antisense
catcaaaggacagccactcc

SEQ ID NO: 18 - DNA, 18 bp, Artificial Sequence
  human GAPDH, sense
gaaggtgaaggtcggagt

SEQ ID NO: 19 - DNA, 19 bp, Artificial Sequence
  human GAPDH, antisense
gaagatggtgatgggtttc

SEQ ID NO: 20 - DNA, 20 bp, Artificial Sequence
  mouse GAPDH, sense
caccttcgatgccggggctg

SEQ ID NO: 21 - DNA, 20 bp, Artificial Sequence
  mouse GAPDH, antisense
ggccatgaggtccaccaccc

SEQ ID NO: 22 - DNA, 18 bp, Artificial Sequence
  human Snai1, sense
gaggcggtggcagactag

SEQ ID NO: 23 - DNA, 19 bp, Artificial Sequence
  human Snai1, antisense
gacacatcggtcagaccag

SEQ ID NO: 24 - DNA, 19 bp, Artificial Sequence
  mouse Snai1, sense
ccactgcaaccgtgctttt

SEQ ID NO: 25 - DNA, 18 bp, Artificial Sequence
  mouse Snai1, antisense
tcttcacatccgagtggg

SEQ ID NO: 26 - DNA, 20 bp, Artificial Sequence
  SBE1 F
ccaggttgagccagtattag

SEQ ID NO: 27 - DNA, 19 bp, Artificial Sequence
  SBE1 R
caggcgaacgggtgctaat

SEQ ID NO: 28 - DNA, 19 bp, Artificial Sequence
  SBE2 F
gacacccactagtgcacac

SEQ ID NO: 29 - DNA, 19 bp, Artificial Sequence
  SBE2 R
gaatgccagctcataaacg

SEQ ID NO: 30 - DNA, 19 bp, Artificial Sequence
  SBE3/4 F
cgtttatgagctggcattc

SEQ ID NO: 31 - DNA, 20 bp, Artificial Sequence
  SBE3/4 R
ccatcttctctcaccactcc

SEQ ID NO: 32 - DNA, 19 bp, Artificial Sequence
  SBE5/6 F
ggagtggtgagagaagatg

SEQ ID NO: 33 - DNA, 19 bp, Artificial Sequence
  SBE5/6 R
tccccacacacgggcctct

SEQ ID NO: 34 - DNA, 19 bp, Artificial Sequence
  SBE7 F
cccactgaggacagggatc

SEQ ID NO: 35 - DNA, 19 bp, Artificial Sequence
  SBE7 R
gcccagaagccctgaggag

SEQ ID NO: 36 - DNA, 19 bp, Artificial Sequence
  SBE8 F
gccataaagcatgagagcc

SEQ ID NO: 37 - DNA, 19 bp, Artificial Sequence
  SBE8 R
gagaaagtcagttaccctg

SEQ ID NO: 38 - DNA, 19 bp, Artificial Sequence
  SBE9 F
caaccctcagccagtatcc

SEQ ID NO: 39 - DNA, 21 bp, Artificial Sequence
  SBE9 R
gcatactgtggccactgtgtc

SEQ ID NO: 40 - DNA, 19 bp, Artificial Sequence
  SBE10/11 F
gtaggagtccaacaaaggg

SEQ ID NO: 41 - DNA, 20 bp, Artificial Sequence
  SBE10/11 R
acttcgcctgtgctactgtg

SEQ ID NO: 42 - DNA, 19 bp, Artificial Sequence
  SBE12/13 F
cacagtagcacaggcgaag

SEQ ID NO: 43 - DNA, 19 bp, Artificial Sequence
  SBE12/13 R
gccctcagcaattgagctg

SEQ ID NO: 44 - DNA, 20 bp, Artificial Sequence
  SBE14 F
gcaccctgctccaagagaag

SEQ ID NO: 45 - DNA, 20 bp, Artificial Sequence
  SBE14 R
ggccggcaggattgggccgc

SEQ ID NO: 46 - DNA, 20 bp, Artificial Sequence
  SBE15 F
ctggctgccggtctaggagg

SEQ ID NO: 47 - DNA, 20 bp, Artificial Sequence
  SBE15 R
ccaggcacgcagagacttgg

SEQ ID NO: 48 - DNA, 20 bp, Artificial Sequence
  SBE16 F
gaatcggcccaaacaaaccc

SEQ ID NO: 49 - DNA, 19 bp, Artificial Sequence
  SBE16 R
gagctccaggtgcagaaac

SEQ ID NO: 50 - DNA, 22 bp, Artificial Sequence
  P13 sense
gtcagcaggcagagtcacttgc

SEQ ID NO: 51 - DNA, 30 bp, Artificial Sequence
  P13 antisense
ctaggcaagtgactctgcctgctgacagct

SEQ ID NO: 52 - DNA, 22 bp, Artificial Sequence
  P14 sense
gggcaaaagacagaaagtaacc

SEQ ID NO: 53 - DNA, 30 bp, Artificial Sequence
  P14 antisense
ctagggttactttctgtcttttgcccagct

SEQ ID NO: 54 - DNA, 22 bp, Artificial Sequence
  P15 sense
gtgcaccagcagaggctggggc

SEQ ID NO: 55 - DNA, 30 bp, Artificial Sequence
  P15 antisense
ctaggccccagcctctgctggtgcacagct

SEQ ID NO: 56 - DNA, 22 bp, Artificial Sequence
  Scramble P14 sense
gggcaaatggcgaaaagtaacc

SEQ ID NO: 57 - DNA, 30 bp, Artificial Sequence
  Scramble P14 antisense
ctagggttacttttcgccatttgcccagct